(19) United States
(12) Patent Application Publication
(10) Pub. No.: US 2026/0035349 A1
(43) Pub. Date: Feb. 5, 2026

(54) 3,4-alkoxy Substituted Phenethyl Lactam Compound, Preparation Method therefor, and Use Thereof

(71) Applicants: Guangzhou Chia Tai Innovative Pharmaceutical Co., Ltd., Guangzhou (CN); Institute of Materia Medica Chinese Academy of Medical Sciences, Beijing (CN)

(72) Inventors: Chen Wang, Beijing (CN); Xiang Zhang, Beijing (CN); Fei Xiao, Beijing (CN); Yonghong Liao, Beijing (CN); Xu Yang, Beijing (CN); Min Dong, Beijing (CN); Dali Yin, Beijing (CN); Xiaomao Xu, Beijing (CN); Qiong Xiao, Beijing (CN); Yulin Tian, Beijing (CN); Hong Yang, Beijing (CN)

(73) Assignees: Guangzhou Chia Tai Innovative Pharmaceutical Co., Ltd., Guangzhou (CN); Institute of Materia Medica Chinese Academy of Medical Sciences, Beijing (CN)

(21) Appl. No.: 19/277,665

(22) Filed: Jul. 23, 2025

Related U.S. Application Data
(60) Provisional application No. 63/678,667, filed on Aug. 2, 2024.

(51) Int. Cl. C07D 237/14; A61K 31/4015; A61K 31/4412; A61K 31/45; A61K 31/50; A61K 31/513; C07D 207/26; C07D 211/76; C07D 211/94; C07D 213/64; C07D 213/89; C07D 239/36

(52) U.S. Cl. CPC C07D 237/14; A61K 31/4015; A61K 31/4412; A61K 31/45; A61K 31/50; A61K 31/513; C07D 207/26; C07D 211/76; C07D 211/94; C07D 213/64; C07D 213/89; C07D 239/36

(57) ABSTRACT

The present disclosure pertains to the technical field of pharmaceutics, and relates to a 3,4-alkoxy substituted phenethyl lactam compound as an enzyme inhibitor of phosphodiesterases, a preparation method therefor, and pharmaceutical use thereof. Specifically, the present disclosure relates to a compound represented by general formula (I), a preparation method therefor, and use thereof in a medicament for preventing and/or treating a phosphodiesterase-associated disease.

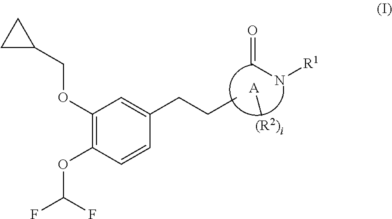

### CROSS-REFERENCE TO RELATED APPLICATION

The application claims priority to and the benefit of U.S. provisional Application No. 63/678,667 filed on Aug. 2, 2024 in the USPTO, the disclosure of which is incorporated herein in its entirety by reference.

### TECHNICAL FIELD

The present disclosure pertains to the technical field of pharmaceutics, and relates to a 3,4-alkoxy substituted phenethyl lactam compound as an enzyme inhibitor of phosphodiesterases, a preparation method therefor, and pharmaceutical use thereof. Specifically, the present disclosure relates to compounds represented by general formula (I), a preparation method therefor, and use thereof in a medicament for preventing and/or treating a phosphodiesterase-associated disease.

### BACKGROUND

Chronic obstructive pulmonary disease (COPD) is a common chronic inflammatory disease of the respiratory system characterized by persistent inflammatory symptoms of the respiratory system and airflow limitation due to airway and/or alveolar abnormalities, which is generally associated with exposure to toxic particles and gases. Common symptoms of COPD include dyspnea, cough, expectoration, and the like. In 2015, an estimated 3.17 million people died globally from chronic obstructive pulmonary disease, accounting for 5% of all deaths worldwide in the same year. In 2016, there were a total of 251 million cases of chronic obstructive pulmonary disease globally. By 2030, COPD will become the third leading cause of death worldwide. The first-line drugs for treating COPD are mainly various types of bronchodilators, including short-acting and long-acting β2 agonists, short-acting and long-acting anticholinergic drugs, combined use of β2 agonists and anticholinergic drugs, the oral drugs methylxanthine and roflumilast, and combined use of glucocorticoids and long-acting β2 agonists. However, there is still no clinical evidence that existing drug therapies can alleviate the long-term trend of pulmonary function decline in patients suffering from COPD. Therefore, effective anti-inflammatory treatment of patients suffering from COPD, which is a chronic inflammatory disease, should be able to slow the progression and exacerbation of the disease and reduce mortality and the incidence of COPD-associated comorbidities.

Cyclic adenosine monophosphate (cAMP) is an important intracellular second messenger, and is involved in physiological activities such as visual conduction, cell proliferation and differentiation, gene expression, inflammatory responses, apoptosis, and cell metabolism, and the like, mainly by activating the PKA pathway. The elevation of the intracellular cAMP level can regulate a variety of inflammatory mediators, thereby achieving a broad-spectrum anti-inflammatory effect. The balance of CAMP in vivo is mainly regulated by adenyl cyclase (CA) and phosphodiesterases (PDEs), in which CA catalyzes the synthesis of cAMP from ATP, and PDEs promote the degradation of cAMP into 5′-AMP, an inactive product. Therefore, intracellular cAMP levels can be elevated to achieve the broad-spectrum anti-inflammatory purpose by the targeted inhibition of the activity of PDEs, among which PDE4 is a central cAMP degradation enzyme. Roflumilast, the first selective PDE4 inhibitor used for the treatment of COPD, was originally developed by Nycomed and was approved in Europe and Canada in 2010 for the adjuvant treatment of patients suffering from frequent exacerbation of severe chronic obstructive pulmonary disease. However, its dose-dependent side effects, such as gastrointestinal adverse effects, e.g., nausea, vomiting, and the like, are severe enough to reduce its compliance. There is accumulating evidence that the adverse effects such as nausea, vomiting, and the like caused by the effects of PDE4 inhibitors exerted on central system PDE4 have greatly limited the wide clinical use of such drugs.

### SUMMARY

The present disclosure relates to a 3,4-alkoxy substituted phenethyl lactam compound as an enzyme inhibitor of phosphodiesterases, a preparation method therefor, and use thereof. Specifically, the present disclosure relates to a compound represented by general formula (I), a preparation method therefor, and use thereof in a medicament for preventing and/or treating a phosphodiesterase-associated disease.

Specifically, the present disclosure relates to a 3,4-alkoxy substituted phenethyl lactam compound of general formula (I), or an isomer or a pharmaceutically acceptable salt thereof:

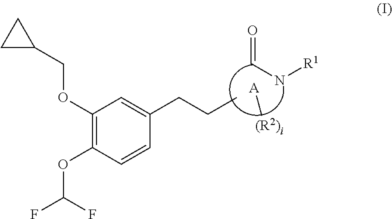

wherein:
ring A is selected from the group consisting of a 5-7 membered lactam ring, and is optionally substituted with R¹ and R²;
R¹ and R² are identical or different and are each independently selected from the group consisting of hydrogen, C₁-C₄ linear or branched alkyl, C₁-C₄ heteroalkyl, C₃-C₆ cycloalkyl, —OR₃, and —(CH₂)zCHF₂;
R³ is selected from the group consisting of hydrogen, C₁-C₄ linear or branched alkyl, C₁-C₄ heteroalkyl, and C₃-C₆ cycloalkyl;
i is selected from the group consisting of 0, 1, 2, 3, and 4; and
z is selected from the group consisting of 0, 1, 2, and 3.

The compound of general formula (II), or the isomer, or the pharmaceutically acceptable salt thereof:

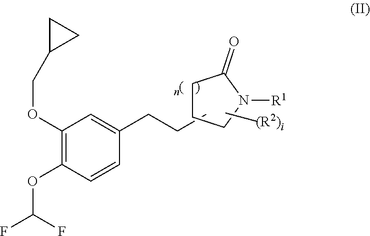

wherein the nitrogen and carbonyl containing ring is optionally substituted with R¹ and R², wherein R¹ and R² are identical or different and are each independently selected from the group consisting of hydrogen, C₁-C₄ linear and branched alkyl, C₁-C₄ heteroalkyl, C₃-C₆ cycloalkyl, —OR³, and —(CH₂)zCHF₂; R³ is selected from the group consisting of hydrogen, C₁-C₄ linear or branched alkyl, C₁-C₄ heteroalkyl, and C₃-C₆ cycloalkyl; i is selected from the group consisting of 0, 1, 2, 3, and 4; z is selected from the group consisting of 0, 1, 2, and 3; n is selected from the group consisting of 1, 2, and 3.

For the compound of general formula (I), when ring A is selected from the group consisting of a 5-7 membered saturated lactam ring, ring A is optionally substituted with R¹ and R²; preferably, 2-(cyclopropylmethoxy)-1-(difluoromethoxy)-4-phenethyl is located meta or para to the carbonyl group on the 5-7 membered lactam ring substituted with R¹ and R², wherein R¹ and R² on the lactam ring are identical or different and are each independently selected from the group consisting of hydrogen, C₁-C₄ linear or branched alkyl, C₁-C₄ heteroalkyl, C₃-C₆ cycloalkyl, —OR³, and —(CH₂)zCHF₂; R³ is selected from the group consisting of hydrogen, C₁-C₄ linear or branched alkyl, C₁-C₄ heteroalkyl, and C₃-C₆ cycloalkyl; i is selected from the group consisting of 0, 1, 2, 3, and 4; z is selected from the group consisting of 0, 1, 2, and 3; n is selected from the group consisting of 1, 2, and 3.

For the compound of general formula (I), when ring A is selected from the group consisting of 2-pyridone, 2-pyrazinone, 2-pyrimidinone, 3-pyridazinone, 4-pyrimidinone, and triazinone, ring A is optionally substituted with R¹ and R², wherein R¹ and R² are independently selected from the group consisting of hydrogen, C₁-C₄ linear or branched alkyl, C₁-C₄ heteroalkyl, C₃-C₆ cycloalkyl, —OR³, and —(CH₂)zCHF₂; R³ is selected from the group consisting of hydrogen, C₁-C₄ linear or branched alkyl, C₁-C₄ heteroalkyl, and C₃-C₆ cycloalkyl; i is selected from the group consisting of 0, 1, 2, and 3; z is selected from the group consisting of 0, 1, 2, and 3.

For the compound of general formula (I), when ring A is selected from the group consisting of 2-pyridone, 2-pyrazinone, 2-pyrimidinone, 3-pyridazinone, 4-pyrimidinone, or triazinone, ring A is optionally substituted with R¹ and R²; preferably, 2-(cyclopropylmethoxy)-1-(difluoromethoxy)-4-phenethyl is located meta or para to the carbonyl group on the 5-6 membered lactam ring substituted with R¹ and R², wherein R¹ and R² are independently selected from the group consisting of hydrogen, C₁-C₄ linear or branched alkyl, C₁-C₄ heteroalkyl, C₃-C₆ cycloalkyl, —OR³, and —(CH₂)zCHF₂; R³ is selected from the group consisting of hydrogen, C₁-C₄ linear or branched alkyl, C₁-C₄ heteroalkyl, and C₃-C₆ cycloalkyl; i is selected from the group consisting of 0, 1, 2, and 3; z is selected from the group consisting of 0, 1, 2, and 3.

The present disclosure provides a compound of formula (I) above, or an isomer, or a pharmaceutically acceptable salt thereof, wherein the compound is selected from:

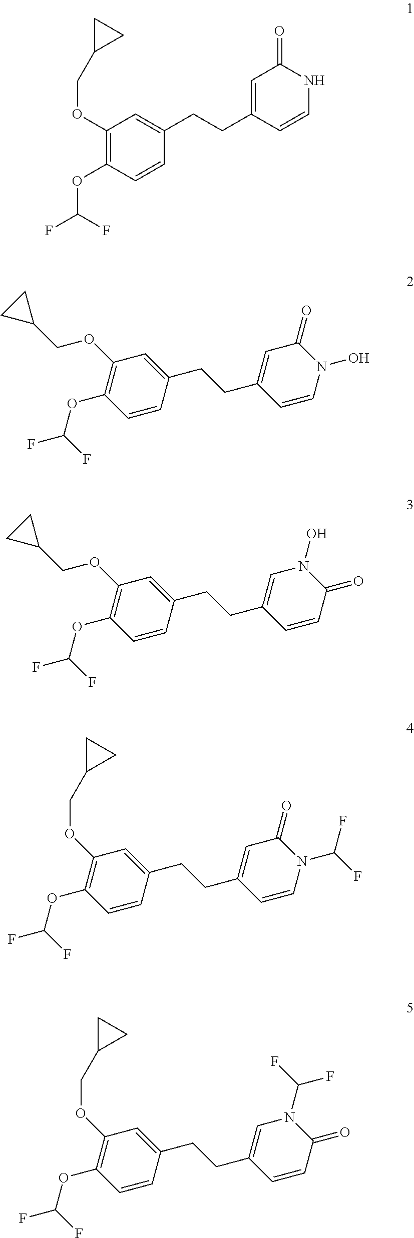 [SMILES: O=c1cc(CCc2ccc(OC(F)F)c(OCC3CC3)c2)cc[nH]1 ; O=c1cc(CCc2ccc(OC(F)F)c(OCC3CC3)c2)ccn1O ; O=c1ccc(CCc2ccc(OC(F)F)c(OCC3CC3)c2)cn1O ; O=c1cc(CCc2ccc(OC(F)F)c(OCC3CC3)c2)ccn1C(F)F ; O=c1ccc(CCc2ccc(OC(F)F)c(OCC3CC3)c2)cn1C(F)F]

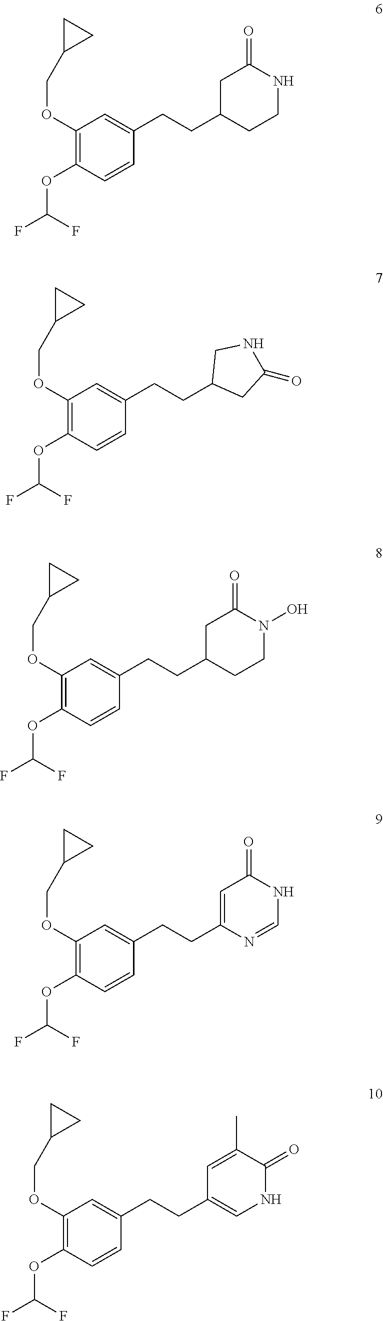 [SMILES: O=C1CC(CCc2ccc(OC(F)F)c(OCC3CC3)c2)CCN1 ; O=C1CC(CCc2ccc(OC(F)F)c(OCC3CC3)c2)CN1 ; O=C1CC(CCc2ccc(OC(F)F)c(OCC3CC3)c2)CCN1O ; O=c1cc(CCc2ccc(OC(F)F)c(OCC3CC3)c2)nc[nH]1 ; Cc1cc(CCc2ccc(OC(F)F)c(OCC3CC3)c2)c[nH]c1=O]

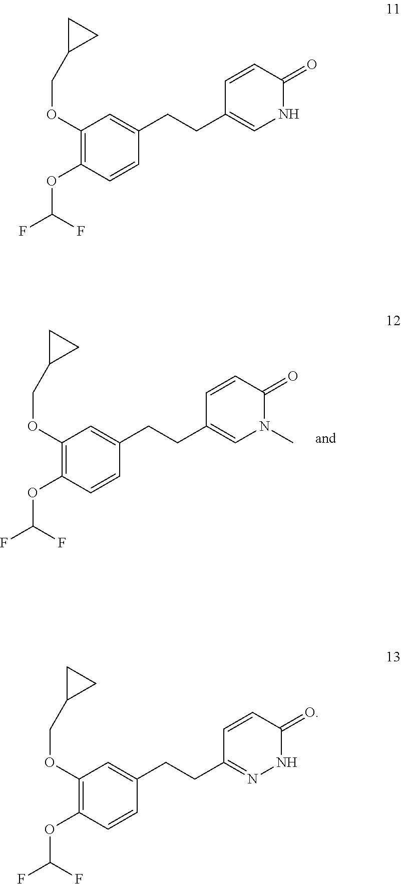 [SMILES: O=c1ccc(CCc2ccc(OC(F)F)c(OCC3CC3)c2)c[nH]1 ; Cn1cc(CCc2ccc(OC(F)F)c(OCC3CC3)c2)ccc1=O ; O=c1ccc(CCc2ccc(OC(F)F)c(OCC3CC3)c2)n[nH]1]

The C₁-C₄ linear or branched alkyl described above is methyl, ethyl, n-propyl, isopropyl, n-butyl, isobutyl, sec-butyl, or tert-butyl.

The C₁-C₄ heteroalkyl described above is linear or branched alkyl containing one N or O atom, such as methoxy, ethoxy, n-propoxy, isopropoxy, n-butoxy, isobutoxy, sec-butoxy or tert-butoxy, methylamino, ethylamino, n-propylamino, isopropylamino, n-butylamino, isobutylamino, or sec-butylamino or tert-butylamino.

The C₃-C₆ cycloalkyl described above is selected from:

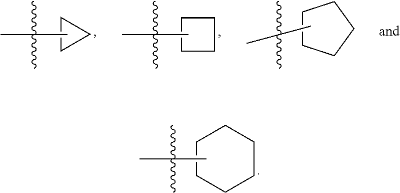

A pharmaceutical combination, comprising an effective dose of the compound of general formula (I), or the isomer, or the pharmaceutically acceptable salt thereof, and a pharmaceutically acceptable carrier or excipient.

The present disclosure provides use of a compound of general formula (I) or an isomer, or a pharmaceutically acceptable salt thereof, as an inhibitor of phosphodiesterases, for preparing a medicament for preventing and/or treating a phosphodiesterase-associated disease.

The present disclosure provides a method for preventing and/or treating a phosphodiesterase-associated disease in a subject in need thereof, comprising administering to the subject an effective dose of the compound of general formula (I) or an isomer or a pharmaceutically acceptable salt thereof.

Studies have found that the compound of general formula (I), within a nM range, exhibits inhibitory activity against the PDE4B1 enzyme in vitro and inhibitory activity against TNF-α release at the cellular level. Most preferably, in vitro pharmacokinetic study, compound 1 shows a relatively short half-life that is better than that of roflumilast, and in vivo pharmacodynamic study, shows a relatively good anti-inflammatory effect. The drug also has a relatively strong affinity for lung tissue and can be retained in the lungs for a long time to exert its drug effect, which is suggesting the feasibility of pulmonary administration.

The compound of general formula (I) may be prepared according to conventional methods known in the art. Some methods that can be used are described below, and they should not be construed as limiting the scope of synthetic methods that can be used to prepare the compound of the present disclosure.

The preparation method for the compound of general formula (I) is as follows:

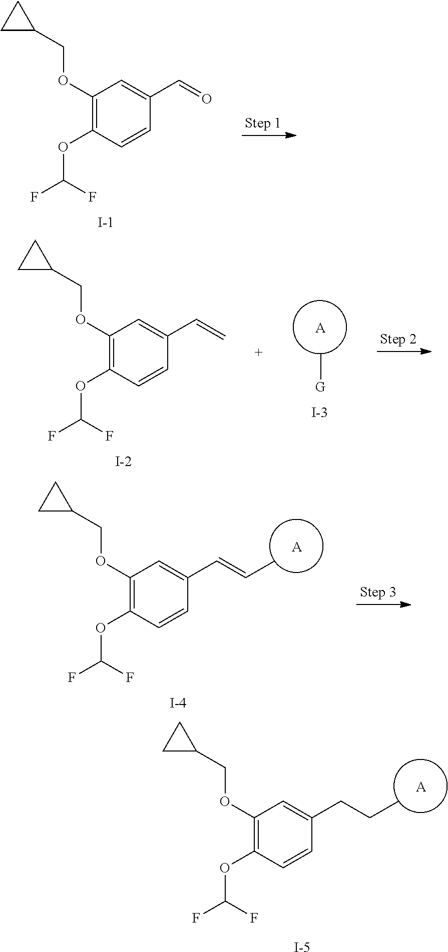

wherein the first step is a compound of formula I-1 reacting with a Wittig salt under alkaline conditions to yield compound I-2;
the second step is compounds I-2 and I-3 undergoing a Heck reaction under alkaline conditions in the presence of a catalyst to give a compound of formula I-4;
the third step is reducing compound I-4 in the presence of a catalyst to give compound I-5.

There are some special target compounds in the examples, and their synthetic methods involve obtaining the target compounds from compound I-5 by functional group derivatization or other synthesis methods, which can be illustrated according to specific steps in the subsequent examples and is not specifically and exhaustively illustrated here; only a general synthetic route is detailed here.

Compounds that provide the alkaline conditions are, but are not limited to, triethylamine, potassium tert-butoxide or sodium tert-butoxide, and sodium hydroxide.

The catalysts are, but are not limited to, palladium/carbon, Raney nickel, tetrakis(triphenylphosphine)palladium(0), tris(dibenzylideneacetone)dipalladium(0), or palladium acetate.

The solvents are, but are not limited to: methanol, ethanol, tetrahydrofuran, dichloromethane, 1,4-dioxane, or N,N-dimethylformamide.

In the preparation method:
G is a leaving group and is selected from the group consisting of halogen and hydroxyl, preferably bromine;
ring A is as defined in general formula (I).

The present disclosure provides use of a compound of general formula (I) or an isomer, or a pharmaceutically acceptable salt thereof, as an inhibitor of phosphodiesterases, for preparing a medicament for preventing and/or treating any disease by inhibiting phosphodiesterases.

Beneficial technical effects: In the present disclosure, several representative compounds with novel structures are obtained by precisely modifying the structure of a 3,4-alkoxy substituted phenethyl lactam compound, and in vitro enzymatic activity detection experiments, the compounds show relatively good bioactivity and in the enzymological and cellular activity research, their activity is 10 nmol or lower. Compound 1 has a short half-life, has a certain anti-inflammatory effect, that shows a good research prospect.

### BRIEF DESCRIPTION OF THE DRAWINGS

FIG. 1: The specificity of compound 1 was determined under HPLC conditions, and peak shapes and symmetry were examined. The retention time of compound 1 was about 7.3 min, and the peak shape was symmetrical without tailing, which can meet the requirements of chromatographic analysis.

FIG. 2: The linearity of compound 1 was determined under HPLC conditions and plotted with the abscissa representing concentration (C) and the ordinate representing peak area (A), a regression calculation was performed, a regression curve was plotted, and a regression equation was obtained. The compound 1 sample exhibited a good linear relationship within the range of 100 ng/ml-2000 ng/mL.

FIG. 3: The concentration-dependent relationship of compound 1 adsorption in ex vivo lung tissue was determined and plotted with the abscissa representing concentration (C) and the ordinate representing the lung tissue affinity index (the ratio of the drug concentration in lung tissue to the drug concentration in free liquid). The lung tissue affinity index showed an upward trend as the concentration of compound 1 increased.

FIG. 4: The desorption rate of compound 1 in ex vivo tissue was determined and plotted with the abscissa representing time points and the ordinate representing tissue concentration. Compound 1 exhibited a relatively slow desorption rate on ex vivo lung tissue, indicating that the drug has a relatively good affinity for lung tissue.

### DETAILED DESCRIPTION

The present disclosure is further described below with reference to examples; however, these examples are not intended to limit the scope of the present disclosure.

### Examples

The structures of the compounds were determined by nuclear magnetic resonance (NMR) spectroscopy or/and mass spectrometry (MS). The NMR analysis was performed using Mercury-400 and BrukerAVAVCE III 500 nuclear magnetic resonance systems, with deuterated dimethyl sulfoxide (DMSO-d₆), deuterated chloroform (CDCl₃), and deuterated methanol (CD₃OD) as solvents and tetramethylsilane (TMS) as an internal standard.

The MS analysis was performed using a Thermo Exactive Orbitrap plus mass spectrometer (ESI-MS).

The thin-layer chromatography silica gel plates used were Qingdao GF254 silica gel plates, and the column chromatography silica gel was Qingdao Haiyang 300-400 mesh silica gel.

The known starting materials in the present disclosure may be synthesized in-house using known methods or purchased commercially.

In the examples, unless otherwise specified, all the reactions can be performed in an argon atmosphere or nitrogen atmosphere.

The argon atmosphere or nitrogen atmosphere means that the reaction flask is connected to a balloon containing about 1 L of argon or nitrogen gas.

The hydrogen atmosphere means that the reaction flask is connected to a balloon containing about 1 L of hydrogen gas.

In the examples, unless otherwise specified, the reaction temperature was room temperature, which is 20-30° C.

In the examples, the monitoring of reaction progress used thin-layer chromatography developing systems, including: A: a dichloromethane and methanol system, and B: a petroleum ether and ethyl acetate system. The volume ratio of the solvents was adjusted according to the polarity of the compounds, or a small amount of basic or acidic reagents such as triethylamine and acetic acid could be added for adjustment.

### Example 1

Preparation of 2-(cyclopropylmethoxy)-1-(difluoromethoxy)-4-ethenylbenzene (1-a)

Methyltriphenylphosphonium iodide (33.4 g, 64 mmol) and potassium tert-butoxide (9.3 g, 64 mmol) were placed in a three-necked flask, and anhydrous THF (100 mL) was added. The mixture was reacted at room temperature for 1 hour in an argon atmosphere. 4-(Difluoromethoxy)-3-(cyclopropylmethoxy)benzaldehyde (7.7 g, 32 mmol) was dissolved in anhydrous THF (20 mL), and the solution was added slowly and dropwise to the reaction solution via a constant-pressure dropping funnel. After the dropwise addition, the mixture was reacted for another 3 hours. Most THF was removed by concentration under reduced pressure, and a saturated ammonium chloride solution (30 mL) was added to quench the reaction. The reaction mixture was extracted three times with ethyl acetate (3×50 mL), and the organic phases were combined and washed once with a saturated aqueous sodium chloride solution. The organic phase was isolated, dried over anhydrous sodium sulfate, and separated by medium-pressure column chromatography to give a colorless transparent oil (1-a, yield: 92%).

¹H NMR (400 MHz, CDCl₃) δ 7.11 (d, J=8.2 Hz, 1H), 6.99 (d, J=2.0 Hz, 1H), 6.96 (dd, J=8.2, 2.0 Hz, 1H), 6.65 (dd, J=12, 20 Hz, 1H), 6.62 (t, J=76.0 Hz, 1H), 5.68 (dd, J=17.5, 0.8 Hz, 1H), 5.25 (dd, J=10.8, 0.8 Hz, 1H), 3.89 (d, J=6.9 Hz, 2H), 1.34-1.26 (m, 1H), 0.68-0.63 (m,2H), 0.38-0.34 (m, 2H); HRMS (ESI): (M+H)⁺ 241.1030.

### Example 2

Preparation of (E)-1-benzyl-4-(3-(cyclopropylmethoxy)-4-(difluoromethoxy)styryl)pyridin-2(1H)-one (1-b)

1-a (1.1 g, 4.56 mmol), 1-benzyl-4-bromopyridin-2(1H)-one (1.2 g, 4.56 mmol), Pd₂(dba)₃ (42 mg, 0.0456 mmol), HP(t-Bu)₃BF₄ (53 mg, 0.1824 mmol), and DABCO (1.53 g, 13.68 mmol) were added in sequence to a sealed tube, and anhydrous dioxane (15 mL) was added. The system was purged with argon, and the stopper was tightened. The mixture was reacted at 100° C. for 36 hours. After the reaction solution was cooled to room temperature, a saturated aqueous sodium bicarbonate solution (10 mL) was added to quench the reaction. The reaction mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a white solid (1-b, yield: 52.40%).

Mp: 100-103° C.;

1H NMR (400 MHz, Chloroform-d) δ 7.36-7.26 (m, 5H), 7.21 (d, J=7.1 Hz, 1H), 7.13 (d, J=8.0 Hz, 1H), 7.08-6.98 (m, 3H), 6.83-6.31 (m, 4H), 5.12 (s, 2H), 3.91 (d, J=6.9 Hz, 2H), 1.32-1.27 (m, 1H), 0.67-0.63 (m, 2H), 0.38-0.34 (m, 2H). ¹³C NMR (100 MHz, Chloroform-d) δ 161.42, 137.00, 135.70, 135.45, 129.06, 128.34, 128.25, 123.24, 110.66, 51.79. HRMS (ESI): (M+H)⁺424.1732.

### Example 3

Preparation of 4-(3-(cyclopropylmethoxy)-4-(difluoromethoxy)phenethyl)pyridin-2(1H)-one (1)

1-b (70 mg, 0.17 mmol) was dissolved in methanol (20 mL), and palladium on carbon (20 mg) was added. The mixture was reacted at 90° C. for 5 h in a hydrogen atmosphere. The palladium on carbon was filtered out with diatomite, and the diatomite layer was washed with ethyl acetate (2×10 mL). The organic phases were combined and separated by medium-pressure column chromatography to give a white solid (47 mg, yield: 83.75%).

Mp: 114-117° C.;

¹H NMR (500 MHz, Chloroform-d) δ 7.29 (d, J=6.4 Hz, 1H), 7.06 (d, J=8.0 Hz, 1H), 6.71-6.74 (m, 2H), 6.58 (t, J=75.5 Hz, 1H), 6.12 (d, J=6.4 Hz, 1H) 3.83 (d, J=6.9 Hz, 2H), 2.87-2.75 (m, 4H), 1.77 (brs, 1H), 1.38-1.15 (m, 1H), 0.64 (t, J=6.7 Hz, 2H), 0.35 (t, J=5.1 Hz, 2H). ¹³C NMR (100 MHz, Chloroform-d) δ 150.45, 139.18, 138.94, 122.78, 120.87, 118.92, 116.35, 114.70, 73.96, 37.33, 35.24, 10.20, 3.20. HRMS (ESI): (M+H)⁺ 336.1414.

### Example 4

Preparation of (E)-4-(3-(cyclopropylmethoxy)-4-(difluoromethoxy)styryl)-2-methoxy-pyridine (2-a)

This example adopted the synthetic method for 1-b and used 2-methoxy-4-bromopyridine as a substrate, and a white solid (380 mg, yield: 53%) was obtained.

Mp: 128-130° C.;

¹H NMR (400 MHz, CDCl₃) δ 8.14 (d, J=5.4 Hz, 1H), 7.19 (d, J=10.5 Hz, 1H), 7.16 (d, J=2.9 Hz, 1H), 7.08 (d, J=6.9 Hz, 2H), 7.00 (d, J=5.5 Hz, 1H), 6.90 (d, J=16.2 Hz, 1H), 6.78 (s, 1H), 6.65 (t, J=76.0 Hz, 1H), 3.97 (s, 3H), 3.93 (d, J=6.9 Hz, 2H), 1.36-1.28 (m, 1H), 0.70-0.62 (m, 2H), 0.40-0.36 (m, 2H); HRMS (ESI): (M+H)⁺ 348.1398.

### Example 5

Preparation of 4-(3-(cyclopropylmethoxy)-4-(difluoromethoxy)phenethyl)-2-methoxy-pyridine (2-b)

This example adopted the synthetic method for 1 and used 2-a as a substrate, and a colorless transparent oil (150 mg, yield: 60%) was obtained.

¹H NMR (400 MHz, CDCl₃) δ 8.05 (dd, J=5.3, 0.7 Hz, 1H), 7.06 (d, J=8.1 Hz, 1H), 6.72 (dd, J=8.1, 2.0 Hz, 1H), 6.68 (dd, J=5.0, 1.6 Hz, 2H), 6.58 (t, J=76.0 Hz, 1H), 6.53 (s, 1H), 3.92 (s, 3H), 3.80 (d, J=6.9 Hz, 2H), 2.87-2.84 (m, 4H), 1.28-1.20 (m, 2H), 0.65-0.60 (m, 2H), 0.35-0.31 (m, 2H); HRMS (ESI): (M+H)⁺ 350.1577.

### Example 6

Preparation of 4-(3-(cyclopropylmethoxy)-4-(difluoromethoxy)phenethyl)-2-methoxy-pyridine 1-oxide (2-c)

2-b (130 mg, 0.37 mmol) was dissolved in anhydrous dichloromethane (20 mL), and m-chloroperoxybenzoic acid (40 mg, 1.1 mmol) was added. A drying tube was mounted on top of the single-necked flask, and the mixture was reacted overnight at room temperature and separated by medium-pressure column chromatography to give a white solid (60 mg, yield: 44%).

Mp: 100-102° C.;

¹H NMR (400 MHz, CDCl₃) δ 8.19 (d, J=6.6 Hz, 1H), 7.07 (d, J=8.0 Hz, 1H), 6.72 (dd, J=6.7, 2.3 Hz, 1H), 6.70-6.64 (m, 2H), 6.59 (t, J=76.0 Hz, 1H), 6.54 (d, J=2.2 Hz, 1H), 3.98 (s, 3H), 3.82 (d, J=6.9 Hz, 2H), 2.95-2.85 (m, 4H), 1.29-1.29 (m, 1H), 0.68-0.60 (m, 2H), 0.37-0.31 (m, 2H); HRMS (ESI): (M+H)⁺ 366.1515.

### Example 7

Preparation of 4-(3-(cyclopropylmethoxy)-4-(difluoromethoxy)phenethyl)-1-hydroxy-pyridin-2(1H)-one (2)

2-c (130 mg, 0.36 mmol) was dissolved in acetyl chloride (6 mL), and the mixture was heated at reflux for 1 hour. The reaction mixture was cooled to room temperature and concentrated under reduced pressure to remove excess acetyl chloride. Methanol was subsequently added, and the mixture was reacted overnight at room temperature. Methanol was removed by concentration under reduced pressure, and the residue was slurried with diethyl ether to give a white solid (80 mg, yield: 64%).

Mp: 149-151° C.;

¹H NMR (400 MHz, CDCl₃) δ 7.66 (d, J=7.1 Hz, 1H), 7.06 (d, J=8.7 Hz, 1H), 6.75-6.67 (m, 2H), 6.59 (t, J=76.0 Hz, 1H), 6.52 (d, J=2.1 Hz, 1H), 6.11 (dd, J=7.1, 2.2 Hz, 1H), 3.83 (d, J=6.9 Hz, 2H), 2.85 (dd, J=8.5, 5.9 Hz, 2H), 2.78 (dd, J=8.5, 5.9 Hz, 2H), 1.30-1.22 (m, 1H), 0.66-0.61 (m, 2H), 0.36-0.32 (m, 2H); ¹³C NMR (100 MHz, CDCl₃) δ 157.67, 152.73, 150.47, 138.99 (t, J=3 Hz), 138.93, 130.75, 122.80, 120.86, 116.31 (t, J=258 Hz), 116.12, 114.69, 107.22, 77.24, 73.96, 36.99, 35.65, 10.18, 3.19; HRMS (ESI): (M+H)⁺ 352.1357.

### Example 8

Preparation of (E)-5-(3-(cyclopropylmethoxy)-4-(difluoromethoxy)styryl)-2-methoxy-pyridine (3-a)

This example adopted the synthetic method for 1-b and used 2-methoxy-5-bromopyridine as a substrate, and a white solid (480 mg, yield: 66%) was obtained.

Mp: 131-133° C.;

¹H NMR (400 MHz, CDCl₃) δ 8.22 (t, J=2.2 Hz, 1H), 7.78 (dt, J=8.7, 2.6 Hz, 1H), 7.14 (dd, J=8.1, 1.4 Hz, 1H), 7.08-7.01 (m, 2H), 6.99-6.86 (m, 2H), 6.76 (dd, J=8.7, 2.3 Hz, 1H), 6.64 (t, J=76.0 Hz, 1H), 3.97 (d, J=2.1 Hz, 3H), 3.93 (dd, J=6.9, 1.4 Hz, 2H), 1.37-1.27 (m, 1H), 0.69-0.64 (m, 2H), 0.40-0.36 (m, 2H); HRMS (ESI): (M+H)⁺ 348.1397.

### Example 9

Preparation of 5-(3-(cyclopropylmethoxy)-4-(difluoromethoxy)phenethyl)-2-methoxy-pyridine (3-b)

This example adopted the synthetic method for 1, and a colorless transparent oil (180 mg, yield: 72%) was obtained.

¹H NMR (400 MHz, CDCl₃) δ 7.91 (d, J=2.4 Hz, 1H), 7.32 (dd, J=8.5, 2.5 Hz, 1H), 7.05 (d, J=8.0 Hz, 1H), 6.70 (dd, J=8.1, 2.0 Hz, 1H), 6.67-6.64 (m, 2H), 6.58 (t, J=76.0 Hz, 1H), 3.90 (s, 3H), 3.80 (d, J=6.9 Hz, 2H), 2.82 (s, 4H), 1.28-1.18 (m, 1H), 0.65-0.60 (m, 2H), 0.35-0.31 (m, 2H); HRMS (ESI): (M+H)⁺ 350.1573.

### Example 10

Preparation of 5-(3-(cyclopropylmethoxy)-4-(difluoromethoxy)phenethyl)-2-methoxy-pyridine 1-oxide (3-c)

This example adopted the synthetic method for 2-c, and a white solid product (100 mg, yield: 64%) was obtained.

Mp: 130-132° C.;

¹H NMR (400 MHz, CDCl₃) δ 8.13 (d, J=2.1 Hz, 1H), 7.06 (d, J=8.3 Hz, 1H), 6.98 (dd, J=8.5, 2.1 Hz, 1H), 6.78 (d, J=5.7 Hz, 1H), 6.67 (d, J=6.6 Hz, 2H), 6.58 (t, J=76.0 Hz, 1H), 4.05 (s, 3H), 3.82 (d, J=6.9 Hz, 2H), 3.09-2.58 (m, 4H), 1.32-1.20 (m, 1H), 0.66-0.61 (m, 2H), 0.36-0.32 (m, 2H); ¹³C NMR (100 MHz, CDCl₃) δ 157.13, 150.52, 139.71, 138.73, 131.18, 128.35, 122.81, 120.95, 116.30 (t, J=258 Hz), 114.78, 107.58, 77.25, 73.98, 57.27, 36.57, 33.66, 10.18, 3.19; HRMS (ESI): (M+H)⁺ 366.1508.

### Example 11

Preparation of 5-(3-(cyclopropylmethoxy)-4-(difluoromethoxy)phenethyl)-1-hydroxy-pyridin-2(1H)-one (3)

This example adopted the synthetic method for 2, and a white solid product (100 mg, yield: 69%) was obtained.

Mp: 170-172° C.;

¹H NMR (400 MHz, CDCl₃) δ 7.51 (d, J=2.4 Hz, 1H), 7.19 (dd, J=9.2, 2.4 Hz, 1H), 7.08 (d, J=8.1 Hz, 1H), 6.71-6.64 (m, 3H), 6.59 (t, J=76.0 Hz, 1H), 3.82 (d, J=6.9 Hz, 3H), 2.81 (ddd, J=9.1, 6.9, 1.9 Hz, 2H), 2.72 (ddd, J=9.1, 6.9, 1.9 Hz, 2H), 1.30-1.20 (m, 1H), 0.66-0.62 (m, 2H), 0.36-0.32 (m, 2H); ¹³C NMR (100 MHz, CDC13) 8 156.67, 150.48, 139.01, 138.97, 138.94, 129.54, 122.85, 120.98, 119.32, 117.19, 116.30 (t, J=258 Hz), 114.83, 77.24, 73.97, 36.79, 33.43, 10.18, 3.20; HRMS (ESI): (M+H)⁺ 352.1351.

### Example 12

Preparation of (E)-N-(4-(3-(cyclopropylmethoxy)-4-(difluoromethoxy)styryl)pyridin-2-yl)acetamide (4-a)

This example adopted the synthetic method for 1-b and used 2-acetamido-4-bromopyridine as a substrate, and a white solid (560 mg, yield: 72%) was obtained.

Mp: 202-204° C.;

¹H NMR (400 MHz, CDCl₃) δ 8.34 (s, 1H), 8.20 (d, J=5.3 Hz, 1H), 7.30 (s, 1H), 7.25 (s, 1H), 7.17 (d, J=8.2 Hz, 1H), 7.15-7.11 (m, 2H), 7.10 (dd, J=8.2, 2.0 Hz, 1H), 6.98 (d, J=16.3 Hz, 1H), 6.66 (t, J=75.4 Hz, 1H), 3.94 (d, J=7.0 Hz, 2H), 2.24 (s, 3H), 1.38-1.26 (m, 1H), 0.70-0.66 (m, 2H), 0.42-0.38 (m, 2H); HRMS (ESI): (M+H)⁺ 375.1511.

### Example 13

Preparation of (E)-4-(3-(cyclopropylmethoxy)-4-(difluoromethoxy) styryl)-1-(difluoromethyl)pyridin-2(1H)-one (4-b)

4-a (130 mg, 0.35 mmol) was dissolved in acetonitrile (20 mL), and sodium chlorodifluoroacetate (64 mg, 0.42 mmol) and 18-crown-6 (19 mg, 0.07 mmol) were added. The mixture was refluxed for 5 hours in an argon atmosphere. The reaction mixture was cooled to room temperature. An equal volume of a 1% aqueous potassium hydrogen sulfate solution was added, and the mixture was refluxed for 2 hours. The reaction mixture was then cooled to room temperature and concentrated under reduced pressure to remove the organic solvent. 0.5 M hydrochloric acid was added, and the mixture was extracted three times with ethyl acetate (3×15 mL). The organic phases were combined and washed once with a saturated aqueous sodium chloride solution (20 mL). The organic phase was isolated, dried over anhydrous sodium sulfate, and separated by medium-pressure column chromatography to give a white solid (80 mg, yield: 60%).

Mp: 151-153° C.;

¹H NMR (400 MHz, CDCl₃) δ 7.68 (t, J=60.3 Hz, 1H), 7.43 (d, J=7.5 Hz, 1H), 7.20-7.07 (m, 4H), 6.79 (d, J=16.3 Hz, 1H), 6.66 (t, J=76 Hz, 1H), 6.52 (d, J=8 Hz, 2H), 6.49 (s, 1H), 3.93 (d, J=6.8 Hz, 2H), 1.38-1.28 (m, 1H), 0.70-0.65 (m, 2H), 0.40-0.37 (m, 2H); ¹³C NMR (100 MHz, CDCl₃) δ 150.83, 149.05, 141.33, 134.89, 134.07, 128.94 (t, J=3 Hz), 125.34, 122.89, 120.78, 118.50, 116.02 (t, J=259 Hz), 112.67, 107.48 (t, J=249 Hz), 104.43, 77.24, 74.13, 10.16, 3.27; HRMS (ESI): (M+H)⁺ 384.1219.

### Example 14

Preparation of 4-(3-(cyclopropylmethoxy)-4-(difluoromethoxy)phenethyl)-1-(difluoro-methyl)pyridin-2(1H)-one (4)

This example adopted the synthetic method for 1, and a solid (90 mg, yield: 44%) was obtained.

Mp: 100-102° C.;

¹H NMR (400 MHz, CDCl₃) δ 7.65 (t, J=60.3 Hz, 1H), 7.35 (d, J=7.3 Hz, 1H), 7.08 (d, J=8.6 Hz, 1H), 6.75-6.67 (m, 2H), 6.59 (t, J=76.0 Hz, 1H), 6.33 (s, 1H), 6.11 (dd, J=7.4, 1.7 Hz, 1H), 3.83 (d, J=6.9 Hz, 2H), 2.85 (dd, J=8.8, 5.9 Hz, 2H), 2.74 (dd, J=9.2, 6.3 Hz, 2H), 1.30-1.22 (m, 1H), 0.66-0.61 (m, 2H), 0.36-0.32 (m, 2H); ¹³C NMR (100 MHz, CDCl₃) δ 161.14, 155.94, 150.52, 139.02 (t, J=3 Hz), 138.78, 128.68 (t, J=3 Hz), 122.87, 120.82, 118.88, 116.30 (t, J=258 Hz), 114.67, 108.94, 107.46 (t, J=249 Hz), 77.25, 73.98, 37.13, 34.73, 10.18, 3.18; HRMS (ESI): (M+H)⁺ 386.1368.

### Example 15

### Preparation of (E)-N-(5-(3-(cyclopropylmethoxy)-4-(difluoromethoxy)styryl)pyridin-2-yl)acetamide (5-a)

This example adopted the synthetic method for 1-b and used 2-acetamido-4-bromopyridine as a substrate, and a product (700 mg, yield: 90%) was obtained.

¹H NMR (400 MHz, CDCl₃) δ 8.34 (d, J=2.3 Hz, 1H), 8.23 (d, J=9.0 Hz, 2H), 7.88 (dd, J=8.7, 2.4 Hz, 1H), 7.15 (d, J=8.1 Hz, 1H), 7.07-7.05 (m, 2H), 6.98 (q, J=16.4 Hz, 2H), 6.64 (t, J=75.5 Hz, 1H), 3.93 (d, J=6.9 Hz, 2H), 2.23 (s, 3H), 1.37-1.27 (m, 1H), 0.69-0.65 (m, 2H), 0.40-0.36 (m, 2H); HRMS (ESI): (M+H)⁺ 375.1514.

### Example 16

### Preparation of (E)-5-(3-(cyclopropylmethoxy)-4-(difluoromethoxy)styryl)-1-(difluoromethyl)pyridin-2(1H)-one (5-b)

This example adopted the synthetic method for 4-b, and a solid (80 mg, yield: 60%) was obtained.

Mp: 149-151° C.;

¹H NMR (400 MHz, CDCl₃) δ 7.72 (dd, J=9.8, 2.5 Hz, 1H), 7.70 (t, J=60.1 Hz, 1H), 7.47 (d, J=2.5 Hz, 1H), 7.14 (d, J=8.1 Hz, 1H), 7.04-6.98 (m, 2H), 6.77 (dd, J=22.5, 16.4 Hz, 2H), 6.64 (t, J=75.6 Hz, 1H), 6.62 (d, J=9.8 Hz, 1H), 3.92 (d, J=6.9 Hz, 2H), 1.36-1.28 (m, 1H), 0.69-0.65 (m, 2H), 0.39-0.35 (m, 2H); ¹³C NMR (100 MHz, CDCl₃) δ 160.45, 150.77, 140.25 (t, J=3 Hz), 138.24, 135.14, 127.50, 127.24 (t, J=3 Hz), 122.98, 122.95, 122.07, 119.52, 117.99, 116.17 (t, J=259 Hz), 111.91, 107.48 (t, J=250 Hz), 77.25, 74.03, 10.18, 3.25; HRMS (ESI): (M+H)⁺ 384.1218.

### Example 17

Preparation of 5-(3-(cyclopropylmethoxy)-4-(difluoromethoxy)phenethyl)-1-(difluoromethyl)pyridin-2(1H)-one (5)

This example adopted the synthetic method for 1, and a solid (110 mg, yield: 54%) was obtained. Mp: 108-110° C.;

¹H NMR (400 MHz, CDCl₃) δ 7.65 (t, J=60.3 Hz, 1H), 7.21 (dd, J=9.6, 2.5 Hz, 1H), 7.08 (dd, J=5.5, 3.1 Hz, 2H), 6.72-6.67 (m, 2H), 6.58 (t, J=76.0 Hz, 1H), 6.51 (d, J=9.6 Hz, 1H), 3.82 (d, J=6.9 Hz, 2H), 2.80 (dd, J=8.7, 6.5 Hz, 2H), 2.67 (dd, J=8.7, 6.6 Hz, 2H), 1.30-1.20 (m, 1H), 0.66-0.62 (m, 2H), 0.36-0.32 (m, 2H); 13C NMR (100 MHz, CDCl₃) δ 160.60, 150.53, 143.10, 138.95, 126.51 (t, J=3 Hz), 122.91, 121.58, 120.99, 119.55, 116.30 (t, J=258 Hz), 114.81, 107.47 (t, J=249 Hz), 77.24, 73.97, 36.17, 33.65, 10.17, 3.19; HRMS (ESI): (M+H)⁺ 386.1369.

### Example 18

Preparation of 4-(3-(cyclopropylmethoxy)-4-(difluoromethoxy)phenethyl)piperidin-2-one (6)

1 (50 mg, 0.15 mmol) and palladium hydroxide (20 mg) were dissolved in absolute ethanol (10 mL), and the solution was added to a 100 mL single-necked flask and reacted at room temperature for 18 h in a hydrogen atmosphere. The palladium on carbon was filtered out with diatomite, and the diatomite layer was washed with ethyl acetate. The organic phases were combined and separated by medium-pressure column chromatography to give a colorless oil (12 mg, yield: 23.71%).

¹H NMR (500 MHz, Chloroform-d) δ 7.14-6.29 (m, 5H), 3.85 (d, J=6.8 Hz, 2H), 3.45-3.22 (m, 2H), 2.70-2.44 (m, 3H), 2.10-1.75 (m, 4H), 1.63 (dh, J=14.3, 6.7 Hz, 2H), 1.46 (dh, J=10.8, 5.7 Hz, 1H), 0.63 (d, J=7.6 Hz, 2H), 0.34 (q, J=6.2, 5.7 Hz, 2H). ¹³C NMR (125 MHz, Chloroform-d) δ 172.23, 150.49, 140.59, 138.70, 122.76, 120.82, 116.41, 114.62, 73.96, 41.26, 37.82, 37.41, 32.70, 32.05, 28.33, 18.48, 10.25. HRMS (ESI): (M+H)⁺ 340.1708.

### Example 19

Preparation of (3-(cyclopropylmethoxy)-4-(difluoromethoxy)phenyl)methanol (7-a)

4-(Difluoromethoxy)-3-(cyclopropylmethoxy)benzaldehyde (1 g, 4.13 mmol) and sodium borohydride (470 mg, 8.26 mmol) were added to a 100 mL single-necked flask and dissolved in anhydrous methanol (10 mL), and the mixture was reacted at room temperature for 2 h. Water (10 mL) was added. The mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a colorless oil (550 mg, yield: 54.58%).

¹H NMR (500 MHz, Chloroform-d) δ 7.20-6.42 (m, 4H), 4.64 (d, J=11.5 Hz, 2H), 3.87 (dt, J=14.7, 7.4 Hz, 2H), 2.13-1.92 (m, 1H), 1.29 (h, J=7.6 Hz, 1H), 0.65 (dq, J=18.9, 10.1, 8.1 Hz, 2H), 0.35 (tt, J=16.5, 7.9 Hz, 2H). ¹³C NMR (125 MHz, Chloroform-d) δ 150.66, 139.66, 122.74, 119.29, 118.37, 116.31, 114.24, 112.86, 73.90, 64.79, 10.19. HRMS (ESI): (M−H₂O+H)⁺ 227.0868.

### Example 20

Preparation of 4-(bromomethyl)-2-(cyclopropylmethoxy)-1-(difluoromethoxy)benzene (7-b)

7-a (920 mg, 3.77 mmol), carbon tetrabromide (1.5 g, 4.5 mmol), and anhydrous dichloromethane (20 mL) were added to a 100 mL single-necked flask, and triphenylphosphine (1.2 g, 4.5 mmol) was then slowly added at 0° C. The mixture was reacted overnight at room temperature in an argon atmosphere. Water (20 mL) was added to quench the reaction. The mixture was extracted three times with dichloromethane (3×20 mL), and the organic phases were combined and washed once with a saturated aqueous sodium chloride solution (20 mL). The organic phase was isolated, dried over anhydrous sodium sulfate, and separated by medium-pressure column chromatography to give a colorless oil (1.1 g, yield: 95.37%).

¹H NMR (500 MHz, Chloroform-d) δ 7.11 (dd, J=8.3, 4.6 Hz, 1H), 7.03-6.91 (m, 2H), 6.62 (td, J=75.4, 4.6 Hz, 1H), 4.44 (d, J=4.5 Hz, 2H), 3.88 (dd, J=7.0, 4.4 Hz, 2H), 1.29 (s, 1H), 0.66 (p, J=5.2 Hz, 2H), 0.36 (t, J=5.1 Hz, 2H). ¹³C NMR (125 MHz, Chloroform-d) δ 150.63, 140.33, 136.34, 122.82, 121.65, 118.20, 116.13, 114.96, 114.06, 73.94, 33.04, 10.13. HRMS (ESI): (M−Br)⁺ 227.0868.

### Example 21

Preparation of ethyl 5-(3-(cyclopropylmethoxy)-4-(difluoromethoxy)phenyl)-3-oxopentanoate (7-c)

Sodium hydride (30 mg, 1 mmol) and 3 mL of tetrahydrofuran were added to a 50 mL single-necked flask, and ethyl acetoacetate (65 mg, 0.5 mmol) was then added dropwise at 0° C. The mixture was stirred for 30 min. n-Butyllithium (32 mg, 0.5 mmol) was added to the reaction solution, and the mixture was stirred for 30 min. Subsequently, 7-b (150 mg, 0.5 mmol, dissolved in 3 mL of tetrahydrofuran) was added dropwise to the reaction solution, and the mixture was reacted at room temperature for 3 h. Water (10 mL) was added. The mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a colorless oil (100 mg, yield: 57.28%). ¹H NMR (500 MHz, Chloroform-d) δ 7.09 (d, J=8.1 Hz, 1H), 6.91-6.41 (m, 3H), 4.21 (q, J=7.2 Hz, 2H), 3.89 (d, J=6.9 Hz, 2H), 3.46 (s, 2H), 2.91 (d, J=3.6 Hz, 4H), 1.30 (q, J=7.2, 6.4 Hz, 4H), 0.67 (d, J=7.6 Hz, 2H), 0.39 (t, J=5.0 Hz, 2H). ¹³C NMR (125 MHz, Chloroform-d) δ 201.76, 167.11, 150.42, 139.46, 138.69, 122.81, 120.72, 118.40, 116.34, 114.66, 114.27, 73.83, 61.52, 49.45, 44.40, 29.11, 14.11, 10.18. HRMS (ESI): (M+H)⁺ 357.1493.

### Example 22

Preparation of ethyl 5-(3-(cyclopropylmethoxy)-4-(difluoromethoxy)phenyl)-3-hydroxypentanoate (7-d)

This example adopted the synthetic method for 7-a and used 7-c as a starting material, and a colorless oil (45 mg, yield: 74.58%) was obtained.

¹H NMR (500 MHz, Chloroform-d) δ 7.09 (d, J=8.1 Hz, 1H), 6.94-6.32 (m, 3H), 4.21 (q, J=7.2 Hz, 2H), 4.03 (dt, J=8.8, 4.9 Hz, 1H), 3.89 (d, J=6.9 Hz, 2H), 2.82 (ddd, J=14.6, 9.7, 5.3 Hz, 1H), 2.79-2.68 (m, 1H), 2.50 (qd, J=16.6, 6.0 Hz, 2H), 1.92-1.66 (m, 2H), 1.31 (t, J=7.1 Hz, 4H), 0.74-0.59 (m, 2H), 0.38 (d, J=5.1 Hz, 2H). ¹³C NMR (125 MHz, Chloroform-d) δ 173.15, 150.39, 140.66, 122.74, 120.94, 116.43, 114.70, 73.87, 67.06, 60.91, 41.26, 38.08, 31.62, 14.23, 10.24. HRMS (ESI): (M+H)⁺ 359.1637.

### Example 23

Preparation of ethyl 3-bromo-5-(3-(cyclopropylmethoxy)-4-(difluoromethoxy)phenyl)pentanoate (7-e)

7-d (150 mg, 0.44 mmol), anhydrous diethyl ether (4 mL), and pyridine (45 mg, 0.52 mmol) were added to a 50 mL three-necked flask, and phosphorus tribromide (145 mg, 0.52 mmol) was added slowly and dropwise at −20° C. The mixture was stirred for 2 h in an argon atmosphere, then slowly warmed to room temperature, and reacted for 12 h. Water (10 mL) was added. The mixture was extracted three times with ethyl acetate (3×20 mL), and the organic phases were combined and washed once with a saturated aqueous sodium chloride solution (20 mL). The organic phase was isolated, dried over anhydrous sodium sulfate, and concentrated to dryness under reduced pressure to give a light yellow oily crude product (200 mg), and the crude product was directly used in the next step.

### Example 24

Preparation of ethyl 3-cyano-5-(3-(cyclopropylmethoxy)-4-(difluoromethoxy)phenyl)pentanoate (7-f)

7-e (200 mg, 0.48 mmol), 5 mL of acetonitrile, trimethylsilyl cyanide (380 mg, 3.8 mmol), and tetrabutylammonium fluoride (1 g, 3.8 mmol) were added to a 50 mL single-necked flask, and the mixture was refluxed at 90° C. for 12 h. Water (10 mL) was added. The mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a colorless oil (60 mg).

¹H NMR (500 MHz, Chloroform-d) δ 7.13 (d, J=8.1 Hz, 1H), 6.93-6.41 (m, 3H), 4.22 (q, J=7.2 Hz, 2H), 3.90 (d, J=7.0 Hz, 2H), 3.07-2.87 (m, 2H), 2.77 (dt, J=16.0, 6.3 Hz, 2H), 2.60 (dd, J=16.6, 6.9 Hz, 1H), 1.96 (p, J=8.6, 8.0 Hz, 2H), 1.62 (s, 1H), 1.41-1.24 (m, 4H), 0.69 (d, J=7.7 Hz, 2H), 0.40 (d, J=5.1 Hz, 2H). ¹³C NMR (125 MHz, Chloroform-d) δ 169.60, 150.63, 138.94, 138.49, 123.03, 120.78, 116.30, 114.59, 114.24, 73.92, 61.49, 36.67, 33.41, 32.98, 27.05, 14.18, 10.20. HRMS (ESI): (M+H)⁺ 368.1667.

### Example 25

Preparation of 4-(3-(cyclopropylmethoxy)-4-(difluoromethoxy)phenethyl)pyrrolidin-2-one (7)

7-f (40 mg, 0.108 mmol), 3 mL of ethanol, cobalt chloride (30 mg, 0.216 mmol), and sodium borohydride (50 mg, 1.08 mmol) were added to a 50 mL single-necked flask, and the mixture was stirred at 0° C. for 30 min and then reacted at room temperature for 1 h. Water (10 mL) was added. The mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a light yellow solid (15 mg, yield: 42.35%).

Mp: 75-77° C.;

¹H NMR (500 MHz, Chloroform-d) δ 7.10 (d, J=8.0 Hz, 1H), 6.91-6.43 (m, 3H), 6.32 (s, 1H), 3.88 (d, J=6.9 Hz, 2H), 3.54 (t, J=8.4 Hz, 1H), 3.10 (dd, J=9.4, 6.2 Hz, 1H), 2.56 (ddt, J=63.8, 14.7, 7.7 Hz, 4H), 2.09 (q, J=11.1 Hz, 2H), 1.81 (m, 1H), 1.38-1.25 (m, 1H), 0.68 (d, J=7.5 Hz, 2H), 0.38 (d, J=5.1 Hz, 2H). ¹³C NMR (125 MHz, Chloroform-d) δ 178.17, 150.48, 140.16, 122.79, 120.76, 116.34, 114.43, 73.91, 47.99, 36.58, 36.30, 34.35, 33.61, 10.21. HRMS (ESI): (M+H)⁺ 326.1555.

### Example 26

Preparation of 4-(3-(cyclopropylmethoxy)-4-(difluoromethoxy)phenethyl)-1-hydroxypiperidin-2-one (8)

This example adopted the synthetic method for 6 and used 2 as a starting material, and a colorless oil (20 mg, yield: 39.55%) was obtained.

¹H NMR (500 MHz, Chloroform-d) δ 7.10 (d, J=8.1 Hz, 1H), 6.88-6.44 (m, 3H), 6.12 (s, 1H), 3.89 (d, J=6.9 Hz, 2H), 3.48-3.26 (m, 2H), 2.75-2.53 (m, 3H), 2.13-1.79 (m, 5H), 1.76-1.59 (m, 2H), 1.56-1.42 (m, 1H), 0.73-0.62 (m, 2H), 0.38 (t, J=5.2 Hz, 2H). ¹³C NMR (125 MHz, Chloroform-d) δ 150.50, 140.53, 138.71, 122.78, 120.81, 114.62, 73.97, 58.49, 41.34, 37.76, 37.39, 32.69, 32.01, 28.33, 18.48, 10.25, 3.20. HRMS (ESI): (M+H)⁺ 356.1651.

### Example 27

Preparation of (E)-3-benzyl-6-(3-(cyclopropylmethoxy)-4-(difluoromethoxy)styryl)pyrimidin-4(3H)-one (9-a)

1-a (41 mg, 0.17 mmol), 3-benzyl-6-bromopyrimidin-4(3H)-one (50 mg, 0.17 mmol), palladium acetate (1 mg, 0.0017 mmol), tris(o-tolyl)phosphine (2 mg, 0.0068 mmol), and 2 mL of triethylamine were added in sequence to a 38 mL sealed tube. The system was purged with argon, and the stopper was tightened. The mixture was reacted at 100° C. for 12 hours. The reaction solution was cooled to room temperature, and purified water (10 mL) was added to quench the reaction. The reaction mixture was extracted three times with ethyl acetate (3×20 mL), and the organic phases were combined and washed once with a saturated aqueous sodium chloride solution (20 mL). The organic phase was isolated, dried over anhydrous sodium sulfate, and separated by medium-pressure column chromatography to give a light yellow solid (60 mg, yield: 66.7%).

Mp: 156-158° C.;

¹H NMR (400 MHz, Chloroform-d) δ 7.36-7.26 (m, 5H), 7.21 (d, J=7.2 Hz, 1H), 7.14 (d, J=8.1 Hz, 1H), 7.07-7.05 (m, 2H), 7.03-7.02 (m, 1H), 6.77 (d, J=16.3 Hz, 1H), 6.63 (t, J=75.5 Hz, 1H), 6.60 (d, J=2.0 Hz, 1H), 6.35 (dd, J=7.2, 2.0 Hz, 1H), 5.13 (s, 2H), 3.91 (d, J=6.9 Hz, 2H), 1.38-1.27 (m, 1H), 0.71-0.60 (m, 2H), 0.38-0.35 (m, 2H). ¹³C NMR (100 MHz, Chloroform-d) δ 161.47, 158.50, 150.08, 136.41, 135.20, 134.26, 132.99, 132.86, 131.98, 129.16, 128.55, 128.21, 125.44, 124.88, 122.80, 120.97, 116.08, 112.99, 112.25, 74.04, 49.52, 10.16, 3.26. HRMS (ESI): (M+H)⁺ 425.1619.

### Example 28

Preparation of 6-(3-(cyclopropylmethoxy)-4-(difluoromethoxy)phenethyl)pyrimidin-4(3H)-one (9)

This example adopted the synthetic method for 1 and used 9-a as a starting material, and a white solid (20 mg, yield: 33.33%) was obtained.

Mp: 177-180° C.;

¹H NMR (500 MHz, Chloroform-d) δ 7.59 (s, 1H), 7.12 (d, J=7.9 Hz, 1H), 6.85-6.35 (m, 4H), 3.89 (d, J=6.9 Hz, 2H), 2.99-2.86 (m, 4H), 1.36-1.24 (m, 1H), 0.73-0.63 (m, 2H), 0.43-0.35 (m, 2H). ¹³C NMR (100 MHz, Chloroform-d) δ 150.54, 139.03, 138.78, 122.87, 120.87, 116.34, 114.70, 35.19, 10.20, 3.23. MS (ESI): (M+H)⁺ 337.1406.

### Example 29

Preparation of (E)-1-benzyl-5-(3-(cyclopropylmethoxy)-4-(difluoromethoxy)styryl)-3-methylpyridin-2(1H)-one (10-a)

This example adopted the synthetic method for 1-b, and a colorless oil (20 mg, yield: 5.49%) was obtained.

¹H NMR (400 MHz, Chloroform-d) δ 7.59 (dd, J=2.6, 1.3 Hz, 1H), 7.46-7.36 (m, 4H), 7.25 (d, J=2.5 Hz, 1H), 7.18-6.42 (m, 7H), 5.22 (s, 2H), 3.93 (d, J=6.9 Hz, 2H), 2.33-2.19 (m, 3H), 1.42-1.34 (m, 1H), 0.77-0.64 (m, 2H), 0.40 (dt, J=4.8, 1.2 Hz, 2H). ¹³C NMR (100 MHz, Chloroform-d) δ 162.50, 150.69, 139.77, 135.89, 133.67, 133.23, 130.47, 128.95, 128.21, 128.11, 125.22, 124.29, 122.90, 119.11, 116.64, 116.25, 111.76, 73.98, 52.34, 17.72, 10.20, 3.24. HRMS (ESI): (M+H)⁺ 438.1867.

### Example 30

Preparation of 5-(3-(cyclopropylmethoxy)-4-(difluoromethoxy)phenethyl)-3-methylpyridin-2(1H)-one (10)

This example adopted the synthetic method for 1 and used 10-a as a starting material, and a colorless oil (8 mg, yield: 39.82%) was obtained.

¹H NMR (400 MHz, Chloroform-d) δ 7.86-7.39 (m, 2H), 7.16-6.27 (m, 5H), 3.84 (dd, J=6.9, 3.8 Hz, 2H), 2.81 (d, J=3.0 Hz, 3H), 2.58 (tt, J=10.6, 5.5 Hz, 1H), 2.22 (s, 2H), 1.59 (d, J=7.7 Hz, 1H), 1.23 (d, J=7.0 Hz, 3H), 0.71-0.55 (m, 2H), 0.34 (dt, J=4.4, 1.9 Hz, 2H). ¹³C NMR (100 MHz, Chloroform-d) δ 150.68, 145.34, 138.74, 131.59, 126.61, 122.94, 120.96, 116.35, 114.81, 74.07, 36.67, 33.53, 15.79, 10.24. HRMS (ESI): (M+H)⁺ 350.1561.

### Example 31

Preparation of (E)-1-benzyl-5-(3-(cyclopropylmethoxy)-4-(difluoromethoxy)styryl)pyridin-2(1H)-one (11-a)

This example adopted the synthetic method for 1-b, and a colorless oil (30 mg, yield: 5.68%) was obtained.

¹H NMR (400 MHz, Chloroform-d) δ 7.38-7.27 (m, 7H), 7.13-6.27 (m, 7H), 5.15 (d, J=5.9 Hz, 2H), 3.88 (d, J=6.9 Hz, 2H), 1.33-1.25 (m, 1H), 0.75-0.56 (m, 2H), 0.42-0.27 (m, 2H). ¹³C NMR (100 MHz, Chloroform-d) δ 162.06, 150.70, 136.18, 129.02, 129.02, 128.17, 125.70, 123.83, 122.89, 121.56, 119.18, 117.23, 116.23, 111.82, 73.99, 52.01, 29.74, 10.19, 3.24. HRMS (ESI): (M+H)⁺ 424.1729.

### Example 32

Preparation of 5-(3-(cyclopropylmethoxy)-4-(difluoromethoxy)phenethyl)pyridin-2(1H)-one (11)

This example adopted the synthetic method for 1 and used 11-a as a starting material, and a colorless oil (3 mg, yield: 37.88%) was obtained.

¹H NMR (400 MHz, Chloroform-d) δ 7.30 (dd, J=9.3, 2.6 Hz, 1H), 7.11 (d, J=2.7 Hz, 1H), 7.04 (d, J=8.6 Hz, 1H), 6.79-6.34 (m, 4H), 3.81 (d, J=6.9 Hz, 2H), 2.82-2.61 (m, 4H), 1.32-1.16 (m, 1H), 0.67-0.54 (m, 2H), 0.40-0.24 (m, 2H). ¹³C NMR (125 MHz, Chloroform-d) δ 150.43, 143.57, 139.30, 132.54, 122.77, 121.00, 120.10, 119.83, 116.38, 114.82, 73.95, 36.71, 33.38, 10.21, 3.22. HRMS (ESI): (M+H)⁺ 336.1406.

### Example 33

Preparation of (E)-5-(3-(cyclopropylmethoxy)-4-(difluoromethoxy)styryl)-1-methylpyridin-2(1H)-one (12-a)

This example adopted the synthetic method for 9-a, and a colorless oil (40 mg, yield: 12.04%) was obtained.

¹H NMR (500 MHz, Chloroform-d) δ 7.71-7.33 (m, 2H), 7.15 (dd, J=8.7, 5.1 Hz, 1H), 7.01 (d, J=5.3 Hz, 2H), 6.87-6.40 (m, 4H), 4.13-3.83 (m, 2H), 3.67-3.43 (m, 3H), 1.30 -1.27 (m, 1H), 0.69 (t, J=7.0 Hz, 2H), 0.39 (t, J=5.4 Hz, 2H). ¹³C NMR (100 MHz, Chloroform-d) δ 162.54, 150.71, 137.01, 136.39, 135.73, 125.66, 123.74, 122.92, 121.04, 119.11, 116.99, 111.93, 74.00, 37.82, 29.73, 10.20, 3.25. HRMS (ESI): (M+H)⁺ 348.1411.

### Example 34

Preparation of 5-(3-(cyclopropylmethoxy)-4-(difluoromethoxy)phenethyl)-1-methylpyridin-2(1H)-one (12)

This example adopted the synthetic method for 1 and used 12-a as a starting material, and a colorless oil (6 mg, yield: 29.82%) was obtained.

¹H NMR (500 MHz, Methanol-d₄) δ 7.56-7.28 (m, 2H), 7.13-6.95 (m, 1H), 6.92-6.37 (m, 4H), 3.97-3.72 (m, 2H), 3.65-3.42 (m, 3H), 2.94-2.58 (m, 4H), 1.24 (s, 1H), 0.63 (d, J=15.9 Hz, 2H), 0.36 (d, J=14.7 Hz, 2H). ¹³C NMR (125 MHz, Chloroform-d) δ 162.47, 150.45, 141.31, 139.39, 136.18, 122.81, 121.03, 120.44, 118.92, 116.28, 114.85, 73.97, 37.74, 36.83, 33.57, 10.21, 3.24. HRMS (ESI): (M+H)⁺ 350.1562.

### Example 35

Preparation of (E)-2-benzyl-6-(3-(cyclopropylmethoxy)-4-(difluoromethoxy)styryl)pyridazin-3(2H)-one (13-a)

This example adopted the synthetic method for 1-b, and a colorless oil (150 mg, yield: 42.5%) was obtained.

¹H NMR (400 MHz, Chloroform-d) δ 7.53 (d, J=9.7 Hz, 1H), 7.47-7.41 (m, 2H), 7.35-7.27 (m, 3H), 7.18-6.87 (m, 6H), 6.64 (t, J=75.4 Hz, 1H), 5.33 (s, 2H), 3.91 (d, J=6.9 Hz, 2H), 1.29-1.24 (m, 2H), 0.72-0.58 (m, 1H), 0.47-0.28 (m, 2H). ¹³C NMR (100 MHz, Chloroform-d) δ 150.86, 143.82, 136.28, 134.60, 131.60, 130.07, 129.03, 128.69, 128.01, 124.56, 122.93, 120.43, 116.17, 112.19, 55.50, 10.22, 3.31. HRMS (ESI): (M+H)⁺ 425.1675.

### Example 36

Preparation of 6-(3-(cyclopropylmethoxy)-4-(difluoromethoxy)phenethyl)pyridazin-3(2H)-one (13)

This example adopted the synthetic method for 1 and used 13-a as a starting material, and a colorless oil (30 mg, yield: 75.72%) was obtained.

¹H NMR (400 MHz, Chloroform-d) δ 7.06 (dd, J=8.7, 2.7 Hz, 2H), 6.90-6.32 (m, 4H), 3.82 (d, J=6.9 Hz, 2H), 2.89 (tt, J=8.0, 4.0 Hz, 4H), 1.33-1.17 (m, 1H), 0.71-0.53 (m, 2H), 0.44-0.20 (m, 2H). ¹³C NMR (125 MHz, Chloroform-d) δ 150.52, 147.85, 139.14, 134.23, 130.10, 122.86, 120.91, 116.32, 114.75, 73.94, 36.09, 33.99, 10.21, 3.23. HRMS (ESI): (M+H)⁺ 337.1361.

### Example 37

Preparation of (E)-5-(3-(cyclopropylmethoxy)-4-(difluoromethoxy)styryl)-1-methyl-1H-pyrazol-3-amine (14-a)

This example adopted the synthetic method for 9-a, and a light yellow oil (10 mg, yield: 8.74%) was obtained.

¹H NMR (500 MHz, Chloroform-d) δ 7.18-6.39 (m, 7H), 5.79 (d, J=2.1 Hz, 1H), 3.90 (dd, J=6.9, 2.1 Hz, 2H), 3.73 (d, J=2.1 Hz, 3H), 0.95 (td, J=7.5, 2.1 Hz, 1H), 0.65 (h, J=4.7 Hz, 2H), 0.36 (d, J=5.1 Hz, 2H). ¹³C NMR (125 MHz, Chloroform-d) δ 153.46, 150.76, 141.96, 135.25, 131.05, 122.93, 119.53, 116.19, 115.01, 112.56, 89.64, 74.14, 35.78, 10.22, 3.26. HRMS (ESI): (M+H)⁺ 336.1514.

### Pharmacological Experiments

### Assay for Inhibitory Activity Against PDE4B1 at Enzymological Level

The test at the enzymological level used a fluorescence polarization method, and the activity of the synthesized compounds at the enzymological level was evaluated using a PDE4B1 assay kit (BPS Inc.).

### Procedures:

1. 20 μM FAM-Cyclic-3′,5′-AMP was 100-fold diluted to 200 nM with a PDE assay buffer. The unused portion of the stock solution should be aliquoted and stored at −20° C.
2. 1 M DTT was added to the 200 nM FAM-Cyclic-3′,5′-AMP dilution in a ratio of 1:500.
3. The FAM-Cyclic-3′,5′-AMP dilution was added to a test plate at 25 μL/well, except for the blank control wells (Blank).
4. The PDE assay buffer was added to the blank control wells (Blank) at 45 μL/well, and the PDE assay buffer was added to the substrate control wells at 20 μL/well.
5. Working solutions of the compounds were added to the corresponding wells of the test plate at 5 μL/well. The PDE assay buffer (10% DMSO) was added to the positive control wells, the substrate control wells, and the blank control wells (Blank) at 5 μL/well.
6. The PDE4B1 enzyme was thawed on ice in advance, and the completely thawed enzyme was diluted to 1 pg/μL with the PDE assay buffer. The enzyme solution was added to the compound wells and the positive control wells at 20 μL/well. The unused portion of the enzyme stock solution should be aliquoted and stored at −80° C.
7. The test plate was incubated at room temperature for 1 hour with a compound concentration of 10 μM and a DMSO concentration of 1%.
8. The binding agent was diluted with the binding agent diluent (cAMP) in a ratio of 1:100 for later use.
9. After the incubation, the binding agent dilution was added to the test plate at 100 L/well, and the plate was incubated at room temperature for 30 min with slow shaking.
10. After the incubation, an FP assay was performed using Envision with an excitation wavelength of 480 nm and an emission wavelength of 535 nm.
11. The IC₅₀ of the compounds was calculated.

TABLE 1
Enzymological level data for representative compounds
of the present disclosure:

| Com- | | |
|---|---|---|
| pound | | IC₅₀ |
| No. | Compound structure | (nM) |
| 1 | 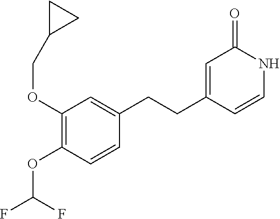 [SMILES: O=c1cc(CCc2ccc(OC(F)F)c(OCC3CC3)c2)cc[nH]1] | 1.3 |
| 2 | 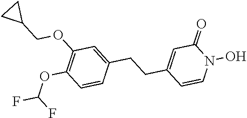 [SMILES: O=c1cc(CCc2ccc(OC(F)F)c(OCC3CC3)c2)ccn1O] | 51.51 |
| 4 | 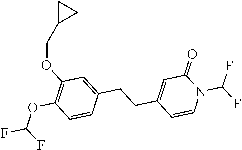 [SMILES: O=c1cc(CCc2ccc(OC(F)F)c(OCC3CC3)c2)ccn1C(F)F] | 755.4 |
| 5 | 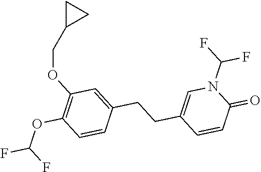 [SMILES: O=c1ccc(CCc2ccc(OC(F)F)c(OCC3CC3)c2)cn1C(F)F] | 299.3 |
| 6 | 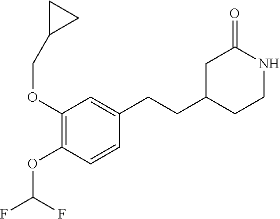 [SMILES: O=C1CC(CCc2ccc(OC(F)F)c(OCC3CC3)c2)CCN1] | 43.15 |
| 7 | 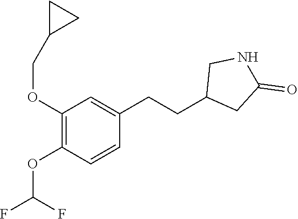 [SMILES: O=C1CC(CCc2ccc(OC(F)F)c(OCC3CC3)c2)CN1] | 80.15 |
| 8 | 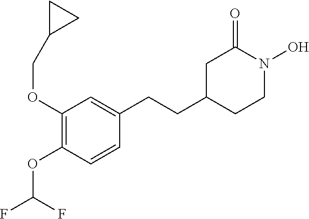 [SMILES: O=C1CC(CCc2ccc(OC(F)F)c(OCC3CC3)c2)CCN1O] | 51.12 |
| 9 | 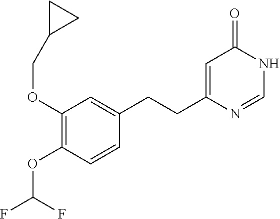 [SMILES: O=c1cc(CCc2ccc(OC(F)F)c(OCC3CC3)c2)nc[nH]1] | 1.96 |
| 10 | 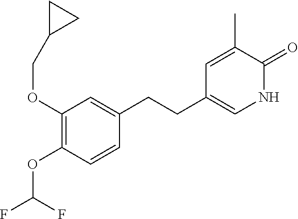 [SMILES: Cc1cc(CCc2ccc(OC(F)F)c(OCC3CC3)c2)c[nH]c1=O] | 298.4 |
| 11 | 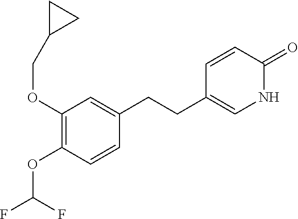 [SMILES: O=c1ccc(CCc2ccc(OC(F)F)c(OCC3CC3)c2)c[nH]1] | 24.97 |
| 12 | 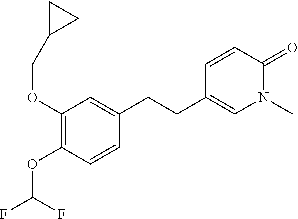 [SMILES: Cn1cc(CCc2ccc(OC(F)F)c(OCC3CC3)c2)ccc1=O] | 81.57 |
| 13 | 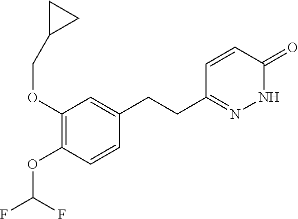 [SMILES: O=c1ccc(CCc2ccc(OC(F)F)c(OCC3CC3)c2)n[nH]1] | 29.31 |

### Evaluation of Inhibitory Activity Against TNF-α Release at Cellular Level

The inhibitory activity of the compounds against the LPS-induced release of TNF-α from PBMCs was evaluated by the Meso Scale Discovery (MSD) method, which is based on electrochemiluminescence technology.

### Procedures:

Day one: thawing of PBMCs

PBMCs were thawed and incubated in an incubator at 37° C. with 5% CO2.

Day two: cell seeding and addition of test compounds and LPS
1. The cells were seeded at a density of 2×105 cells/well in 100 μL of culture medium.
2. For the test compounds, the final test concentrations were 10000, 3333, 1111, 370.33, 123.44, 41.15, 13.72, 4.57, and 1.52 nM.
3. For roflumilast, the final test concentrations were 3000, 1000, 333.33, 111.11, 37.04, 12.35, 4.12, 1.37, and 0.46 nM.
4. LPS was added to each well (with a final concentration of 10 ng/ml).
5. The cells were cultured in an incubator at 37° C. with 5% CO2 for 24 hours.

Day three: detection of human TNF-α
1. The cell supernatant was collected at 100 μL/well.
2. A standard or sample was added to the plate at 50 μL/well, and the plate was sealed with an adhesive plate and incubated at room temperature for 2 hours with shaking.
3. The plate was washed three times.
4. A 1× detection antibody solution was added to each well at 25 μL/well, and the plate was sealed with an adhesive plate and incubated at room temperature for 2 hours with shaking.
5. The plate was washed three times.
6. A 2× reader buffer was added to each well at 150 μL/well, and data were read on the MSD.
   Data analysis:

The assay robustness was assessed using HC and LC:
HC: average signal value of high control group (wells with LPS 10 ng/mL and 0.1% DMSO)
LC: average signal value of low control group (3 μM roflumilast)

$$\% inhibition = ( SignalAve\_HC - Signalcmpd ) / ( SignalAve\_HC - SignalAve\_LC ) \times 100$$

GraphPad Prism 8 software was used to perform calculations and plot dose-response curves of the compounds.

$$Y = Bottom + ( Top - Bottom ) / ( 1 + 1 {0}^{⋀} ( ( LogIC 50 - X ) * HillSlope ) )$$

X: Log of cpd concentration
Y: % inhibition
Top and Bottom: Plateaus in same units as Y
HillSlope: Slope factor or Hill slope

TABLE 2
Cellular-level data for representative
compounds of the present disclosure:

| Compound No. | PBMCs IC₅₀(nM) |
|---|---|
| 1 | 7.849 |
| 2 | 203.6 |
| 3 | 185.7 |
| 9 | 14.91 |

### In Vivo Pharmacokinetic Study

### Procedures:

Three male Sprague-Dawley rats (260-269 g) were housed in a cage and given free access to food and water. The temperature was set to 25° C., and a 12-hour light/dark cycle was maintained. The rats were grouped into one experimental group (n=3): (compound 1). The compound was administered via intravenous injection to each group at 0.3 mg/kg. The compound was dissolved in 10% DMSO/45% PEG400/45% Water. After single administration, blood samples were taken at 5 min, 15 min, 30 min, 1 h, 2 h, 4 h, 8 h, and 24 h. After all blood samples were collected into heparinized test tubes and processed, the compound was analyzed using a validated LC-MS/MS method, and pharmacokinetic parameters (t_{1/2}, Cₘₐₓ, AUC_{0-∞}, AUC_{0-∞}, V_{d}, and CL) were calculated.

TABLE 4
In vivo pharmacokinetic data for representative
compounds of the present disclosure:

| Parameter | Compound 1 |
|---|---|
| Route of administration | Intravenous injection |
| Number of rats/group | 3 |
| Dose of administration (mg/kg) | 0.3 |
| t_{1/2} (h) | 0.321 |
| Tₘₐₓ (h) | 0.0833 |
| Cₘₐₓ (ng/mL) | 292 |
| AUC₀₋ₜ (ng · h/mL) | 152 |
| AUC_{0-∞} (ng · h/mL) | 154 |

### Establishment of In Vitro HPLC Method

### I. Method:

1. Chromatographic conditions:

Wavelength: 210 nm; stationary phase: C18 column; mobile phase: 0.1% phosphoric acid-water solution: acetonitrile; method: 1:1 isocratic elution; temperature: 25° C.; flow rate: 1.0 mL/min; injection volume: 10 μL; run time: 12 min.

### 2. Specificity

5.0 mg of compound 1 powder was brought to volume with methanol in a 5 mL volumetric flask to form a 1 mg/mL stock solution. The stock solution was diluted to different concentrations using acetonitrile, and the test solutions were loaded into liquid phase vials. Samples were injected for HPLC analysis under the chromatographic conditions described above, and peak shapes and symmetry were examined.

### 3. Linearity

Compound 1 was diluted with acetonitrile to a series of concentrations of 10, 20, 50, 100, 200, 500, 1000, 2000, 5000, and 10,000 ng/mL. A 200 μL sample was precisely taken and injected into a liquid chromatograph under the chromatographic conditions described above, and the chromatogram was recorded. Plotting was performed with the abscissa representing concentration (C) and the ordinate representing peak area (A). A regression calculation was performed, a regression curve was plotted, and a regression equation was obtained.

### II. Results

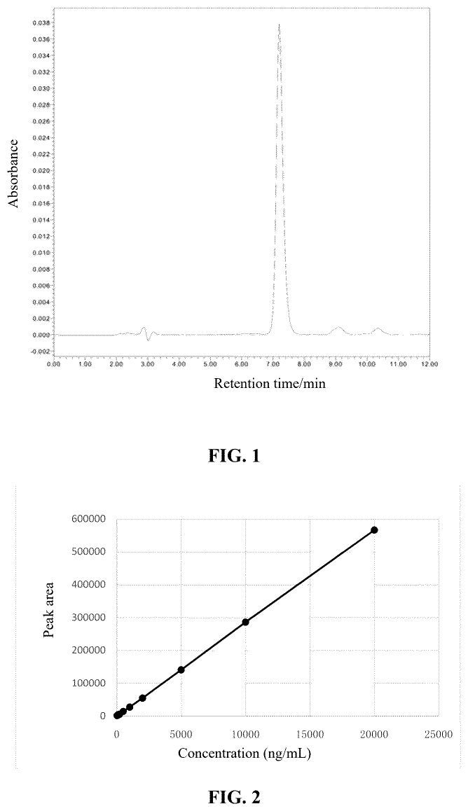

1. Specificity: The retention time of compound 1 was about 7.3 min, and the peak shape was symmetrical without tailing, which can meet the requirements of chromatographic analysis (see FIG. 1).
2. Linearity: The compound 1 sample exhibited a good linear relationship within the range of 100 ng/mL-2000 ng/mL. The linear equation was y=26.867x+542.3, R²=0.9995. The limit of detection was about 10 ng/ml (see FIG. 2).

| Concentration | Peak area of |
|---|---|
| (ng/mL) | compound 1 |
| 20000 | 566126 |
| 10000 | 285408 |
| 5000 | 140259 |
| 2000 | 54444 |
| 1000 | 27043 |
| 500 | 14153 |
| 200 | 5329 |
| 100 | 3838 |
| 50 | 1986 |
| 20 | 1518 |

### Oil-water Partition Coefficient (log P) Test

### I. Method

500 μL of water-saturated n-octanol was added to a glass bottle, and an appropriate amount of solid compound 1 was added. The mixture was ultrasonicated for 1 min, and then 500 μL of water saturated with n-octanol was added. The mixture was ultrasonicated for 1 min, left to stand at room temperature for 24 h, and centrifuged at 12,000 rpm for 10 min. The upper layer solution was carefully pipetted into a new centrifuge tube. Subsequently, the lower layer solution was collected using a pipette and, after the outer wall of the pipette was blotted dry, transferred into a new centrifuge tube. Thus, the upper and lower layer solutions were separated. Subsequently, 10 μL of the upper layer n-octanol solution was collected using a pipette and 50-fold diluted, and 100 μL of the lower layer aqueous solution was collected using a pipette. HPLC analysis was performed.

### II. Experimental Results:

| | Dilution | Original |
|---|---|---|
| | concentration | concentration |
| Peak area | ng/mL | μg/mL |
| 1802301 | 63250.49 | 3162.52 |
| 103738 | 3666.418 | 3.67 |

According to the experimental results, the log P value of compound 1 was 2.94, indicating that it is a poorly soluble compound and is poorly soluble in water.

### Protein Binding Rate Test

### I. Method

The binding rate of compound 1 in plasma was determined by an ultrafiltration method. 50 μL of a 1 mg/mL drug solution of compound 1 was added to 450 μL of fresh rat plasma to form a plasma-drug solution with a final concentration of 100 μg/mL, and the solution was vortexed for 5 min. The solution was centrifuged using an ultrafiltration filter at the rotation speed of 5000 rpm for 15 min, the unbound fraction was collected. After the ultrafiltrate was 5-fold diluted, the concentrations of compound 1 in the bound and unbound fractions were determined by high performance liquid chromatography, as described below. The fraction unbound of the drug and plasma proteins (FP) was calculated using the equation:

$${f}_{p} = \frac{{C}_{u , p}}{{C}_{T} \times ( 1 - Ad )}$$

where C_{u,p} and C_{T} are the unbound concentration and total concentration of the drug in plasma, respectively, and Ad is the non-specific adsorption rate. The binding rate (%) to plasma proteins was calculated using the equation:

$$Binding rate ( \% ) = ( 1 - {f}_{p} ) \times 100$$

### II. Experimental Results

| | | Dilution | Ultrafiltrate | Total | Binding rate |
|---|---|---|---|---|---|
| | Peak | concentration | concentration | concentration | to plasma |
| | area | μg/mL | μg/mL | μg/mL | proteins % |
| Ultrafiltrate | 41211 | 1.4556 | 7.278 | 100 | 92.72 |

### In Vivo Anti-inflammatory Drug Effect (Dose-response Relationship) Test

### I. Method:

1. Preparation of 10 mL of 3% chloral hydrate: 1.2 g of chloral hydrate was dissolved in 40 mL of normal saline.
2. 3 mL of an 80 μg/mL LPS solution was prepared. To prepare a compound 1 suspension, 10 mg of the compound 1 active pharmaceutical ingredient was added to 1 mL of 0.04% Tween 80, and the mixture was ultrasonicated for 20 s in a water bath, vortexed for 5 min, and centrifuged at low speed (200 rpm) for 10 min. The supernatant was collected, and its concentration was determined by HPLC. Then the supernatant was diluted by a corresponding factor to obtain a corresponding concentration. Prepared doses for administration: 2000, 400, 80, and 16 μg/kg, i.e., 800, 160, 32, and 6.4 μg/mL, 1 mL each. (Four concentration groups were set, with n=3)
3. Anesthesia and tracheal administration: 0.22 mL of 3% chloral hydrate was intraperitoneally injected. Balb/c mice were immobilized. The trachea was exposed, and 50 μL of drug solution was collected using a scalp vein set, with an air gap left at the back. The scalp vein set was inserted into the trachea, and administration was performed by fast bolus injection. After the administration, the mice were kept upright for a moment. One hour after the administration, LPS (80 μg/mL, 50 μL) was administered via the trachea. After 6 h, the mice were sacrificed. Bronchoalveolar lavage fluid collection: A blunt needle was inserted into the trachea and fixed, and 0.8 mL of normal saline was injected once, held for 30 s, and withdrawn; the procedure was performed three times. The liquid obtained was then brought to volume (2 mL). After centrifugation at 3000 r/min, the supernatant was collected. 100 μL of PBS was added to the cell pellets, and the cells were counted. Lung tissue was collected and homogenized with 2 mL of normal saline. The homogenate was vortexed for 5 min and centrifuged (12,000 r/min), and the supernatant was collected and analyzed. LPS group: 50 μL of normal saline was administered via the trachea. After 1 h, LPS (80 μg/mL, 50 μL) was administered. After 6 h, collection was performed.

Budesonide positive drug control group: The tracheal administration dose was 0.5 mg/kg (50 μL), and other procedures were the same as those for the compound 1 group.

Cell counting: The cells obtained from the bronchoalveolar lavage fluid by centrifugation were resuspended in 100 μL of PBS. 10 μL of the cell suspension was placed into a cell counting plate and examined under a microscope. The cells in the four primary squares (each was divided into 16 small squares) of the cell counting plate were counted. After counting, the cell count in each mL of the suspension was calculated (cell count in cell suspension/mL=total cell count in 4 primary squares/4×10⁴×dilution factor).

TNF-α was detected using a kit (Bioss).

### II. Results

Cell counting results:

| | Average cell | Average cell |
|---|---|---|
| Group | count in group 1 | count in group 2 |
| Compound 1 800 μg/mL | 6.9*10⁵ | 8.1*10⁵ |
| Compound 1 160 μg/mL | 2.72*10⁶ | 2.38*10⁶ |
| Compound 1 32 μg/mL | 3.68*10⁶ | 2.56*10⁶ |
| Compound 1 6.4 μg/mL | 2.24*10⁶ | 1.92*10⁶ |
| Positive drug group | 3.21*10⁶ | 2.74*10⁶ |
| LPS group | 8.15*10⁶ | 5.26*10⁶ |

The cell counting results show that the cell count in the bronchoalveolar lavage fluid decreased as the concentration of compound 1 increased, indicating that its anti-inflammatory drug effect was enhanced.

### In Vivo Anti-inflammatory Drug Effect (Time-response Relationship) Test

### I. Method:

1. Preparation of 10 mL of 3% chloral hydrate: 1.2 g of chloral hydrate was dissolved in 40 mL of normal saline.
2. 3 mL of a 200 μg/mL LPS solution was prepared.
3. The compound 1 suspension with an HPLC-calibrated concentration was diluted by a corresponding factor to obtain 3 mL of a suspension with a compound 1 concentration of 400 μg/mL. After a dose of 50 μL was administered to each Balb/c mouse, LPS was administered at 12 h, 6 h, and 1 h for modeling (n=3). The mice were sacrificed 6 h after modeling, and the lavage fluid and lung tissue were collected and assayed by following the procedures of the dose-response relationship.

TNF-α was detected using a kit (Bioss).

### II. Results

Cell counting results:

| | Average total |
|---|---|
| | cell count |
| Group | (n = 2-3) |
| 1-h group | 3.0*10⁵ |
| 6-h group | 7.1*10⁵ |
| 12-h group | 3.8*10⁵ |
| LPS group | 4.98*10⁶ |

The above cell counting results show that after prophylactic administration, the cell counts in the compound 1 groups were lower than that in the LPS group, indicating that the drug has a certain anti-inflammatory effect.

TNF-α:

| | Average TNF-α | Average TNF-α |
|---|---|---|
| | content in | content in |
| | bronchoalveolar | lung tissue |
| | lavage fluid | homogenate |
| Group | (pg/mL, n = 2-5) | (pg/mL, n = 3) |
| 1-h group | 105 | 124 |
| 6-h group | 610 | 211 |
| 12-h group | 320 | 134 |
| Normal group | <30 | <30 |
| LPS group | 906 | 261 |

The above TNF-α results show that after prophylactic administration, the TNF-α concentrations in the lavage fluids and lung tissue homogenates in the compound 1 groups were lower than those in the LPS group, indicating that the drug has a certain anti-inflammatory effect.

### Lung Tissue Distribution Test

### I. Method

The compound 1 suspension with an HPLC-calibrated concentration was diluted by a corresponding factor to obtain a suspension with a compound 1 concentration of 400 μg/mL. After administration at a dose of 400 μg/kg, the mice were sacrificed at 30 min, 6 h, 12 h, and 24 h (n=3), and the lung tissue of the mice was collected.

### Establishment of In Vivo Standard Curve of Lung Tissue

Each sample of lung tissue was homogenized with 0.5 mL of normal saline. 100 μL of the homogenate was pipetted into a test tube, and 30 μL of formononetin (internal standard) (original concentration: 5 μg/mL; final concentration: 500 ng/ml), 30 μL of compound 1 (diluted in acetonitrile to different concentrations), and 140 μL of acetonitrile were added. The mixture was vortexed for 5 min and then centrifuged at 12,000 rpm for 15 min, and the supernatant was collected and analyzed by HPLC. Original concentrations: 100 μg/mL, 50 μg/mL, 10 μg/mL, 5 μg/mL, and 1 μg/mL. Final concentrations: 10 μg/mL, 5 μg/mL, 1 μg/mL, 500 ng/mL, and 100 ng/mL.

Sample treatment: The collected fresh lung tissue samples were each homogenized with 0.5 mL of normal saline. 100 μL of the homogenate was pipetted into a test tube, and 30 μL of formononetin (internal standard) and 170 μL of acetonitrile were added. The mixture was vortexed for 5 min and then centrifuged at 12,000 rpm for 15 min, and the supernatant was collected and analyzed by HPLC.

### II. Results

| | | Peak | Peak | | Concentration | Drug | | | Drug | |
|---|---|---|---|---|---|---|---|---|---|---|
| Time of | | area of | area of | Peak | after | concentration | Drug | | amount/ | |
| adminis- | | compound | internal | area | dilution | in lung tissue | amount in | | lung mass | |
| tration | | 1 | standard | ratio | (ng/mL) | (ng/mL) | lung μg | Mean | (μg/g) | Mean |
| 0.5 | h-1 | 17832 | 14557 | 1.22 | 579.32 | 1737.97 | 0.87 | 0.86 | 5.43 | 5.39 |
| 0.5 | h-2 | 17172 | 13998 | 1.23 | 580.17 | 1740.50 | 0.87 | | 5.44 | |
| 0.5 | h-3 | 17490 | 14618 | 1.20 | 565.75 | 1697.24 | 0.85 | | 5.30 | |
| 6 | h-1 | 8491 | 13449 | 0.63 | 296.64 | 889.93 | 0.44 | 0.44 | 2.78 | 2.75 |
| 6 | h-2 | 9316 | 14776 | 0.63 | 296.23 | 888.69 | 0.44 | | 2.78 | |
| 6 | h-3 | 7590 | 12425 | 0.61 | 286.89 | 860.66 | 0.43 | | 2.69 | |
| 12 | h-1 | 4986 | 10890 | 0.46 | 214.02 | 642.07 | 0.32 | 0.41 | 2.01 | 2.54 |
| 12 | h-2 | 7765 | 12562 | 0.62 | 290.35 | 871.05 | 0.44 | | 2.72 | |
| 12 | h-3 | 6555 | 10028 | 0.65 | 307.27 | 921.81 | 0.46 | | 2.88 | |
| 24 | h-1 | 3708 | 11162 | 0.33 | 154.19 | 462.57 | 0.23 | 0.22 | 1.45 | 1.38 |
| 24 | h-2 | 3087 | 11267 | 0.27 | 126.47 | 379.41 | 0.19 | | 1.19 | |
| 24 | h-3 | 4215 | 12052 | 0.35 | 162.54 | 487.62 | 0.24 | | 1.52 | |

The results show that a drug amount of compound 1 was detected in lung tissue within 24 h, indicating that the drug has a relatively strong affinity for lung tissue and can be retained in the lungs for a long time to exert its drug effect.

### Adsorption test in ex vivo lung tissue (concentration dependence)

### I. Method

1. Blank lung preparation: After a rat was sacrificed, its thoracic cavity was opened, and all lung tissue was collected, washed with normal saline to remove the blood on the surface, wrapped in tin paper, and stored at −80° C. for later use.
2. Five 1.5 mL and 2 mL centrifuge tubes were prepared as containers for the unbound drug and lung tissue, respectively, and labeled. 1 mL of acetonitrile was added to each 1.5 mL centrifuge tube. 500 μL of normal saline was added to each 2 mL centrifuge tube.
3. Method of processing lung tissue samples: An appropriate amount of blank lung tissue was thawed at room temperature and cut into small pieces of 1 mm³ with scissors, and each 200 mg of lung tissue was weighed into a small beaker. A total of 5 concentrations were tested, each in triplicate.
4. 5 mL of Ringer buffer was added to each small beaker containing lung pieces, and then 100 μL of a compound 1 drug solution with a concentration of 250 μg/mL (final concentration: 5 μg/mL), 100 μL of a compound 1 drug solution with a concentration of 150 μg/mL (final concentration: 3 μg/mL), 100 μL of a compound 1 drug solution with a concentration of 100 μg/mL (final concentration: 2 μg/mL), 100 μL of a compound 1 drug solution with a concentration of 50 μg/mL (final concentration: 1 μg/mL), and 100 μL of a compound 1 drug solution with a concentration of 25 μg/mL (final concentration: 500 ng/ml) were separately added. Each concentration was tested in triplicate.
5. The small beakers were incubated at 37° C. At 60 min, 500 μL of free liquid was transferred to a 1.5 mL centrifuge tube, and meanwhile, all the lung pieces were transferred to a 2 mL centrifuge tube. After the lung pieces were homogenized, 150 μL of 10 μg/mL formononetin (internal standard) was added, and finally, 850 μL of acetonitrile was added.
6. After 5 min of vortexing, centrifugation (12,000 rpm, 15 min) was performed, and the supernatant was collected. HPLC analysis was performed.
   Conditions: acetonitrile: 0.1% phosphoric acid in water; 1:1 isocratic; 210 nm

### II. Results

| | | Peak | Compound | Drug | Drug | Mass of | Drug |
|---|---|---|---|---|---|---|---|
| Concentration | Peak | area of | 1/internal | concentration | mass in | lung | concentration |
| of compound 1 | area of | internal | standard | in lung extract | lung | piece | in lung |
| (ng/mL) | compound 1 | standard | peak | (ng/mL) | (μg) | (g) | (μg/g) |
| 5000 | 475932 | 92097 | 5.17 | 2827.24 | 4.24 | 0.1945 | 21.80 |
| 5000 | 79823 | 17387 | 4.59 | 2506.81 | 3.76 | 0.1943 | 19.35 |
| 5000 | 409531 | 78821 | 5.20 | 2842.78 | 4.26 | 0.1995 | 21.37 |
| 3000 | 44059 | 16561 | 2.66 | 1434.28 | 2.15 | 0.1933 | 11.13 |
| 3000 | 46823 | 15315 | 3.06 | 1654.79 | 2.48 | 0.2013 | 12.33 |
| 3000 | 189715 | 67159 | 2.82 | 1525.65 | 2.29 | 0.1936 | 11.82 |
| 2000 | 110641 | 55710 | 1.99 | 1059.62 | 1.59 | 0.1958 | 8.12 |
| 2000 | 114020 | 54865 | 2.08 | 1110.83 | 1.67 | 0.2004 | 8.31 |
| 2000 | 42677 | 21659 | 1.97 | 1050.95 | 1.58 | 0.1938 | 8.13 |
| 1000 | 20592 | 18810 | 1.09 | 564.46 | 0.85 | 0.2003 | 4.23 |
| 1000 | 25589 | 25073 | 1.02 | 523.27 | 0.78 | 0.2025 | 3.88 |
| 1000 | 22832 | 21631 | 1.06 | 542.68 | 0.81 | 0.1977 | 4.12 |
| 500 | 7863 | 22334 | 0.35 | 151.87 | 0.23 | 0.1961 | 1.16 |
| 500 | 5976 | 20560 | 0.29 | 117.76 | 0.18 | 0.2000 | 0.88 |
| 500 | 7828 | 21840 | 0.36 | 155.40 | 0.23 | 0.2004 | 1.16 |

| Concentration | Peak area | Detection | Drug | Drug concentration |
|---|---|---|---|---|
| of compound | of | concentration | concentration | in lung/drug |
| 1 added | compound | in free liquid | in free liquid | concentration |
| (ng/mL) | 1 | (ng/mL) | (μg/mL) | in free liquid |
| 5000 | 58534 | 2158.47 | 6.48 | 3.37 |
| 5000 | 41521 | 1525.24 | 4.58 | 4.23 |
| 5000 | 39954 | 1466.92 | 4.40 | 4.86 |
| 3000 | 23518 | 855.16 | 2.57 | 4.34 |
| 3000 | 28465 | 1039.29 | 3.12 | 3.95 |
| 3000 | 26207 | 955.25 | 2.87 | 4.12 |
| 2000 | 17213 | 620.49 | 1.86 | 4.36 |
| 2000 | 17433 | 628.68 | 1.89 | 4.41 |
| 2000 | 28018 | 1022.66 | 3.07 | 2.65 |
| 1000 | 25994 | 947.32 | 2.84 | 1.49 |
| 1000 | 12461 | 443.62 | 1.33 | 2.91 |
| 1000 | 19612 | 709.78 | 2.13 | 1.93 |
| 500 | 8913 | 311.56 | 0.93 | 1.24 |
| 500 | 9694 | 340.63 | 1.02 | 0.86 |
| 500 | 4171 | 135.06 | 0.41 | 2.87 |

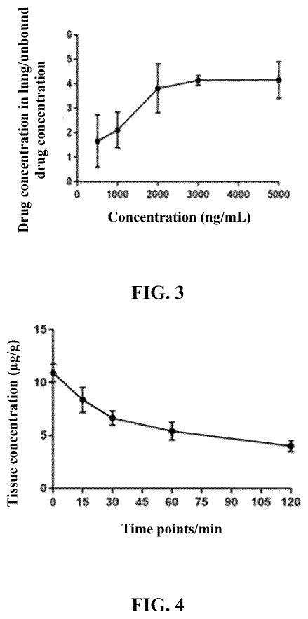

The results show that the adsorption of compound 1 in ex vivo lung tissue exhibited a certain concentration-dependent relationship: the lung tissue affinity index (the ratio of the drug concentration in lung tissue to the drug concentration in free liquid) showed an upward trend as the concentration of compound 1 increased (see FIG. 3). When compound 1 was added at a concentration of 3 μg/mL, the lung tissue affinity index was about 4, indicating a relatively strong affinity for lung tissue.

### Desorption Kinetic Test in Ex Vivo Lung Tissue

### I. Method

1. Blank lung preparation: After a rat was sacrificed, its thoracic cavity was opened, and all lung tissue was collected, washed with normal saline to remove the blood on the surface, and then weighed; that is, blank lung tissue was obtained. The blank lung tissue was wrapped in tin paper and stored at −80° C. for later use.
2. Fifteen 1.5 mL and 2 mL centrifuge tubes were prepared as containers for the unbound drug and lung tissue, respectively, and labeled. 1 mL of acetonitrile was added to each 1.5 mL centrifuge tube. 500 μL of normal saline was added to each 2 mL centrifuge tube.
2. Method of processing lung tissue samples: An appropriate amount of blank lung tissue was thawed at room temperature and cut into small pieces with scissors, and each 200 mg of lung tissue was weighed into a small beaker. The beakers were marked with: 0 min, 15 min, 30 min, 60 min, and 120 min (each in triplicate).
4. 10 mL of Ringer buffer was added to each small beaker, and then 100 μL of a compound 1 drug solution with a concentration of 300 μg/mL was added. (The final concentration was 3 μg/mL)
5. After 1 hour of incubation at 37° C., the lung pieces were transferred to 5 mL of Ringer buffer containing 10% fetal bovine serum and incubated. At 0 min, 15 min, 30 min, 60 min, 120 min, 500 μL of free liquid was transferred to a 1.5 mL centrifuge tube, and meanwhile, all the lung pieces were transferred to a 2 mL centrifuge tube. After homogenization, 150 μL of 10 μg/mL formononetin (internal standard) was added, and finally, 850 μL of acetonitrile was added.
6. After vortexing, centrifugation (12,000 rpm, 15 min) was performed, and the supernatant was collected. HPLC analysis was performed.

### II. Experimental Results

| | | | | | | | Drug content |
|---|---|---|---|---|---|---|---|
| | | Peak | Compound | Drug | Drug | Mass of | in lung/mass |
| | Peak | area of | 1/internal | concentration | mass in | lung | of lung |
| | area of | internal | standard | in lung | lung | piece | piece |
| Time/min | compound 1 | standard | peak | (ng/mL) | (μg) | (g) | (μg/g) |
| 0 | 49805 | 17644 | 2.82 | 1524.48 | 2.29 | 0.1946 | 11.75 |
| 0 | 50603 | 19099 | 2.65 | 1428.23 | 2.14 | 0.1966 | 10.90 |
| 0 | 104260 | 41190 | 2.53 | 1362.50 | 2.04 | 0.2021 | 10.11 |
| 15 | 77008 | 44031 | 1.75 | 927.92 | 1.39 | 0.197 | 7.07 |
| 15 | 27503 | 11815 | 2.33 | 1249.50 | 1.87 | 0.1992 | 9.41 |
| 15 | 84534 | 38708 | 2.18 | 1169.55 | 1.75 | 0.2045 | 8.58 |
| 30 | 67813 | 37283 | 1.82 | 966.76 | 1.45 | 0.1957 | 7.41 |
| 30 | 27105 | 17834 | 1.52 | 800.64 | 1.20 | 0.1945 | 6.17 |
| 30 | 26451 | 16826 | 1.57 | 829.63 | 1.24 | 0.1964 | 6.34 |
| 60 | 25667 | 20531 | 1.25 | 650.81 | 0.98 | 0.2044 | 4.78 |
| 60 | 28262 | 17579 | 1.61 | 849.45 | 1.27 | 0.2002 | 6.36 |
| 60 | 19744 | 15416 | 1.28 | 667.80 | 1.00 | 0.1967 | 5.09 |
| 120 | 15925 | 17066 | 0.93 | 474.69 | 0.71 | 0.2002 | 3.56 |
| 120 | 24896 | 21662 | 1.15 | 594.77 | 0.89 | 0.1983 | 4.50 |
| 120 | 14863 | 14519 | 1.02 | 525.00 | 0.79 | 0.2034 | 3.87 |

The results show that compound 1 exhibited a relatively slow desorption rate on ex vivo lung tissue, indicating that the drug has a relatively good affinity for lung tissue (see FIG. 4). It should be understood that embodiments/examples described herein should be considered in a descriptive sense only and not for purposes of limitation. Descriptions of features or aspects within each embodiment/example should typically be considered as available for other similar features or aspects in other embodiments/examples. While one or more embodiments/examples have been described herein, it will be understood by those of ordinary skill in the art that various suitable changes in form and details may be made therein without departing from the spirit and scope as defined by the claims, and equivalents thereof.

1. A compound, or an isomer, or a pharmaceutically acceptable salt thereof, wherein the compound has the structure of formula (I): 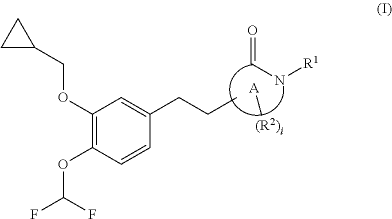
wherein:
ring A is selected from the group consisting of a 5-7 membered lactam ring, and is independently and optionally substituted with R¹ and R²;
R¹ and R² are identical or different and are each independently and optionally selected from the group consisting of hydrogen, C₁-C₄ linear or branched alkyl, C₁-C₄ heteroalkyl, C₃-C₆ cycloalkyl, —OR³, and —(CH₂)_{z}CHF₂;
the R³ described above is selected from the group consisting of hydrogen, C₁-C₄ linear or branched alkyl, C₁-C₄ heteroalkyl, and C₃-C₆ cycloalkyl;
i is selected from the group consisting of 0, 1, 2, 3, and 4;
z is selected from the group consisting of 0, 1, 2, and 3.

2. The compound according to claim 1, or the isomer, or the pharmaceutically acceptable salt thereof: 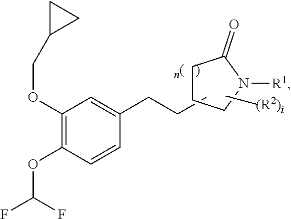
wherein the compound, or the isomer, or the pharmaceutically acceptable salt thereof is independently and optionally substituted with R¹ and R², wherein R¹ and R² are identical or different and are each independently and optionally selected from the group consisting of hydrogen, C₁-C₄ linear or branched alkyl, C₁-C₄ heteroalkyl, C₃-C₆ cycloalkyl, —OR³, and —(CH₂)zCHF₂; the R³ described above is selected from the group consisting of hydrogen, C₁-C₄ linear or branched alkyl, C₁-C₄ heteroalkyl, and C₃-C₆ cycloalkyl; i is selected from the group consisting of 0, 1, 2, 3, and 4; z is selected from the group consisting of 0, 1, 2, and 3; n is selected from the group consisting of 1, 2, and 3.

3. The compound according to claim 2, or the isomer, or the pharmaceutically acceptable salt thereof, wherein 2-(cyclopropylmethoxy)-1-(difluoromethoxy)-4-phenethyl is located meta or para to the carbonyl group on the 5-7 membered lactam ring substituted with R¹ and R², wherein the R¹ and R² on the lactam ring are identical or different and are each independently and optionally selected from the group consisting of hydrogen, C₁-C₄ linear or branched alkyl, C₁-C₄ heteroalkyl, C₃-C₆ cycloalkyl, —OR³, and —(CH₂)_{z}CHF₂; the R³ described above is selected from the group consisting of hydrogen, C₁-C₄ linear or branched alkyl, C₁-C₄ heteroalkyl, and C₃-C₆ cycloalkyl; i is selected from the group consisting of 0, 1, 2, 3, and 4; z is selected from the group consisting of 0, 1, 2, and 3; n is selected from the group consisting of 1, 2, and 3.

4. The compound according to claim 1, or the isomer, or the pharmaceutically acceptable salt thereof, wherein when ring A is selected from the group consisting of 2-pyridone, 2-pyrazinone, 2-pyrimidinone, 3-pyridazinone, 4-pyrimidinone, and triazinone, ring A is independently and optionally substituted with R¹ and R², wherein R¹ and R² are independently and optionally selected from the group consisting of hydrogen, C₁-C₄ linear or branched alkyl, C₁-C₄ heteroalkyl, C₃-C₆ cycloalkyl, —OR³, and —(CH₂)_{z}CHF₂; the R³ described above is selected from the group consisting of hydrogen, C₁-C₄ linear or branched alkyl, C₁-C₄ heteroalkyl, and C₃-C₆ cycloalkyl; i is selected from the group consisting of 0, 1, 2, and 3; z is selected from the group consisting of 0, 1, 2, and 3.

5. The compound according to claim 4, or the isomer, or the pharmaceutically acceptable salt thereof, wherein 2-(cyclopropylmethoxy)-1-(difluoromethoxy)-4-phenethyl is located meta or para to the carbonyl group on the 5-6 membered lactam ring substituted with R¹ and R², wherein R¹ and R² are independently and optionally selected from the group consisting of hydrogen, C₁-C₄ linear or branched alkyl, C₁-C₄ heteroalkyl, C₃-C₆ cycloalkyl, —OR³, and —(CH₂)_{z}CHF₂; the R³ described above is selected from the group consisting of hydrogen, C₁-C₄ linear or branched alkyl, C₁-C₄ heteroalkyl, and C₃-C₆ cycloalkyl; i is selected from the group consisting of 0, 1, 2, and 3; z is selected from the group consisting of 0, 1, 2, and 3.

6. The compound according to claim 1, or the isomer, or the pharmaceutically acceptable salt thereof, wherein the compound is selected from the group consisting of the following compounds:

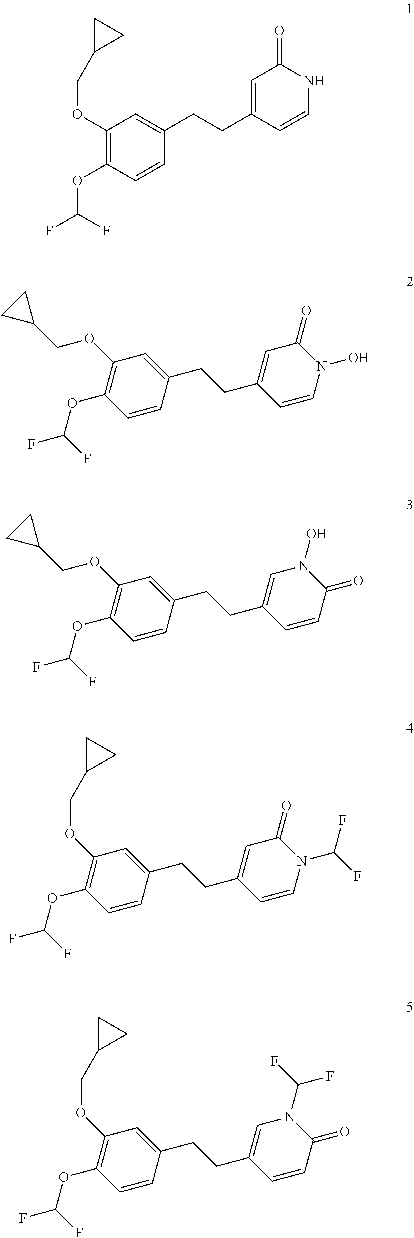 [SMILES: O=c1cc(CCc2ccc(OC(F)F)c(OCC3CC3)c2)cc[nH]1 ; O=c1cc(CCc2ccc(OC(F)F)c(OCC3CC3)c2)ccn1O ; O=c1ccc(CCc2ccc(OC(F)F)c(OCC3CC3)c2)cn1O ; O=c1cc(CCc2ccc(OC(F)F)c(OCC3CC3)c2)ccn1C(F)F ; O=c1ccc(CCc2ccc(OC(F)F)c(OCC3CC3)c2)cn1C(F)F]

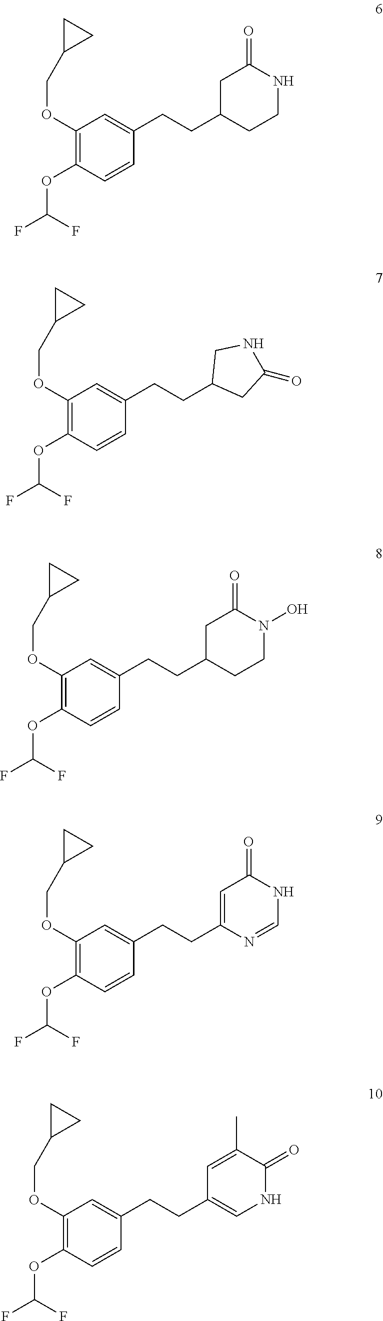 [SMILES: O=C1CC(CCc2ccc(OC(F)F)c(OCC3CC3)c2)CCN1 ; O=C1CC(CCc2ccc(OC(F)F)c(OCC3CC3)c2)CN1 ; O=C1CC(CCc2ccc(OC(F)F)c(OCC3CC3)c2)CCN1O ; O=c1cc(CCc2ccc(OC(F)F)c(OCC3CC3)c2)nc[nH]1 ; Cc1cc(CCc2ccc(OC(F)F)c(OCC3CC3)c2)c[nH]c1=O]

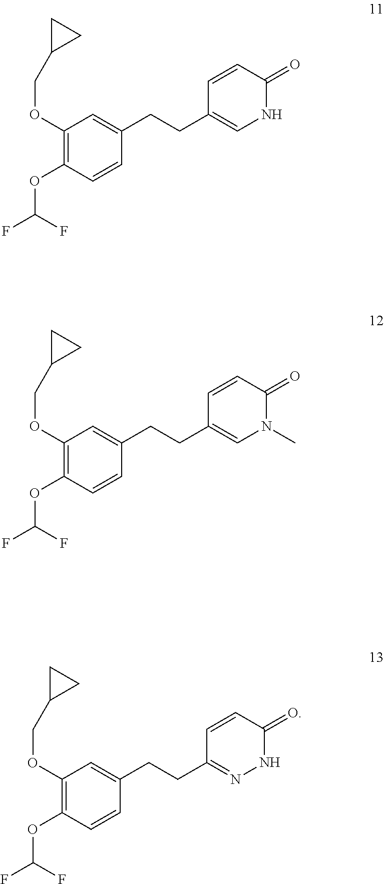 [SMILES: O=c1ccc(CCc2ccc(OC(F)F)c(OCC3CC3)c2)c[nH]1 ; Cn1cc(CCc2ccc(OC(F)F)c(OCC3CC3)c2)ccc1=O ; O=c1ccc(CCc2ccc(OC(F)F)c(OCC3CC3)c2)n[nH]1]

7. A pharmaceutical combination, comprising an effective dose of the compound according to any one of claims 1-6, or the isomer, or the pharmaceutically acceptable salt thereof, and a pharmaceutically acceptable carrier or excipient.

8. Use of the compound according to any one of claims 1-6, or the isomer, or the pharmaceutically acceptable salt thereof for preparing a phosphodiesterase inhibitor.

9. Use of the compound according to any one of claims 1-6, or the isomer, or the pharmaceutically acceptable salt thereof for preparing a medicament for preventing and/or treating a phosphodiesterase-associated disease.
